(19) United States
(12) Patent Application Publication
(10) Pub. No.: US 2023/0355711 A1
(43) Pub. Date: Nov. 9, 2023

(54) VIMENTIN AND FRAGMENTS OR DERIVATIVES THEREOF FOR CORONAVIRUS TREATMENT AND PREVENTION

(71) Applicant: Baylor College of Medicine, Houston, TX (US)

(72) Inventors: Fong Wilson Lam, Houston, TX (US); Miguel Angel Cruz, Pearland, TX (US)

(21) Appl. No.: 17/998,237

(22) PCT Filed: May 14, 2021
(86) PCT No.: PCT/US2021/070560

§ 371 Date: Nov. 8, 2022

Related U.S. Application Data
(60) Provisional application No. 63/024,668, filed on May 14, 2020.

(51) Int. Cl. A61K 38/17; C07K 14/47; A61P 31/14; A61K 45/06

(52) U.S. Cl. CPC A61K 38/1709; C07K 14/47; A61P 31/14; A61K 45/06

(57) ABSTRACT

Embodiments of the disclosure include methods and compositions for treating or preventing coronavirus infection using soluble vimentin of functional variant or derivative thereof. In specific embodiments, a vimentin derivative comprising the rod domain is utilized for treating or preventing any disease in which the blocking of viral particles from binding cell surface receptors is therapeutic. In specific embodiments, a fragment of vimentin that comprises part or all of the rod domain is employed.

This application claims priority to U.S. Provisional Patent Application Ser. No. 63/024,668, filed May 14, 2020, which is incorporated by reference herein in its entirety.

### STATEMENT REGARDING FEDERALLY SPONSORED RESEARCH OR DEVELOPMENT

This invention was made with government support under GM123261, awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jun. 19, 2023, is named BAYM_P0305US_1001232363_SL.txt and is 10,534 bytes in size

### TECHNICAL FIELD

Embodiments of the disclosure include at least the fields of cell biology, molecular biology, and medicine.

### BACKGROUND

In 2019, the Centers for Disease Control and Prevention (CDC) started monitoring the outbreak of a new coronavirus, SARS-CoV-2, which causes COVID-19. SARS-CoV-2, can lead to severe acute respiratory syndrome and life-threatening forms of pneumonia. Authorities first identified the virus in Wuhan, China. Since then, the virus has spread to nearly every country, leading the World Health Organization (WHO) to declare this as a pandemic. As of May 12, 2021, over 160 million people have contracted the virus worldwide, and it has caused over 3.3 million deaths. In the United States alone, the virus has affected over 33 million people, resulting in more than 589,000 deaths.

SARS-CoV-2 infects cells using the spike protein (S), with current reports suggesting angiotensin-converting enzyme 2 (ACE2) on the host cell as the point of contact (1, 2). SARS-CoV-2 S shares ˜76% homology with SARS-CoV S, the spike protein on the coronavirus causing severe acute respiratory syndrome (SARS; SARS-CoV) (3). SARS-CoV S requires binding to both ACE2 and cell surface vimentin to enter cells (4), suggesting that SARS-CoV-2 may also interact with cell surface vimentin to bind to and infect cells.

The present disclosure provides a solution to the need for treatment of certain medical conditions, including at least coronavirus infections such as SARS-CoV-2 that can lead to life-threatening complications, for example.

### BRIEF SUMMARY

Coronavirus binding of cell surface receptors plays a central role in viral infection, leading to morbidity and mortality, and embodiments of the disclosure concern methods and compositions for prevention or treatment thereof. Coronavirus infection can lead to medical conditions including COVID-19, severe acute respiratory syndrome (SARS), or respiratory infections, which can in turn lead to pneumonia, organ failure, respiratory failure, blood clots, heart conditions such as cardiomyopathies, acute kidney injury, and further viral and bacterial infections. Embodiments of the disclosure concern methods and compositions for treating coronavirus infections, including those associated with any medical condition. Particular embodiments of the disclosure utilize vimentin, including soluble vimentin, or functionally active fragments or derivatives thereof (including at least the rod domain or a fragment thereof) for treating coronavirus infections or any medical condition in which the blocking of viral particles from binding cell surface receptors is beneficial. In specific embodiments, the medical condition is treated, or at least one symptom is improved upon, following blockage of coronavirus binding to cell surface receptors.

In specific embodiments, vimentin or functionally active fragments or derivatives thereof results in an improvement of at least one symptom of a medical condition in which coronavirus infection is involved. In particular embodiments, the medical condition is SARS or a respiratory infection. In particular embodiments, SARS or the respiratory infection can lead to life-threatening complications including but not limited to pneumonia, organ failure, blood clots, respiratory failure, heart conditions such as cardiomyopathies, acute kidney injury, or further viral and bacterial infections, for example.

In one embodiment, there is a method of treating or preventing coronavirus infection in an individual, comprising the step of delivering to the individual a therapeutically effective amount of vimentin or a functionally active fragment and/or functionally active variant thereof. In an alternative embodiment, the entire vimentin protein is not utilized because it is unable under particular conditions to bind the spike protein. In another embodiment, there is a composition for treating or preventing coronavirus infection in an individual comprising a therapeutically effective amount of vimentin or a functionally active fragment (such as the rod domain or a fragment thereof) and/or functionally active variant thereof. In some embodiments, the coronavirus is SARS-CoV-2. The coronavirus infection may further comprise SARS or a respiratory infection, and SARS or respiratory infection may comprise pneumonia, organ failure, blood clots, respiratory failure, heart conditions such as cardiomyopathies, acute kidney injury, further viral and bacterial infections, or a combination thereof.

In specific embodiments, blocking any protein or part of SARS-CoV-2 (including the S protein) with recombinant human rod domain of vimentin (rhRod) is useful for preventing or attenuating SARS-CoV-2 infection in an individual. In certain embodiments, vimentin binds a host cell receptor to prevent uptake or infection by the virus, whereas in other embodiments the rod domain of vimentin binds a host cell receptor to prevent uptake or infection by the virus. In specific aspects, vimentin or a functionally active fragment or derivative thereof binds SARS-CoV-2 spike protein to block SARS-CoV-2 spike protein-ACE2 interactions, and in particular cases the rod domain of vimentin interferes with host-virus interactions to result in a therapeutic or preventative effect. In particular cases, the rod domain of vimentin or a functionally active fragment or derivative thereof binds the SARS-CoV-2 spike protein and prevents or reduces infection of SARS-CoV-2 in an individual. In at least some cases, the recombinant human rod domain of vimentin (rhRod) binds the SARS-CoV-2 spike protein.

In specific cases, the fragment of vimentin comprises N-terminal head domain, C-terminal tail domain, the α-helical coiled-coil rod domain, or a combination thereof. The fragment may comprise the N-terminus, the C-terminus, both the N-terminus and C-terminus, or neither of the N-terminus or C-terminus. In some cases, the fragment or derivative is at least 70, 75, 80, 85, 90, 91, 92, 93, 94, 95, 96, 97, 98, or 99% identical to SEQ ID NO:1. In specific embodiments, the variant comprises 1, 2, 3, 4, 5, or more variations compared to SEQ ID NO:1, SEQ ID NO:2, SEQ ID NO:4, or SEQ ID NO:6. In certain cases, the fragment may comprise SEQ ID NO:2, corresponding to the rod domain of vimentin. In some cases, the fragment or derivative is at least 70, 75, 80, 85, 90, 91, 92, 93, 94, 95, 96, 97, 98, or 99% identical to SEQ ID NO:2. In specific embodiments, the variant comprises 1, 2, 3, 4, 5, or more variations compared to SEQ ID NO:2. In some embodiments, the vimentin binds coronavirus spike proteins, thereby preventing interaction of the coronavirus with cell surface receptors.

An individual may be provided a second therapy for the medical condition being treated with vimentin or a fragment or derivative thereof. The second therapy may comprise antibiotics, antivirals, convalescent serum, immune modulators, anticoagulants, fluids, oxygen, a corticosteroid, antibodies, GSnP-6, sialyl Lewis X analog, anti-proliferatives, calcineurin inhibitors, anti-signaling compounds, or a combination thereof.

Any method of the disclosure may further comprise testing for (including resulting in a diagnosis of) a medical condition associated with coronavirus infection or testing for presence of the virus. Any individual may or may not be symptomatic and/or may or may not have been exposed to a coronavirus-infected individual. When an individual is symptomatic, they may or may not have one or more of fever, cough, shortness of breath or difficulty breathing, tiredness, aches, chills, sore throat, loss of smell, loss of taste headache, diarrhea, and vomiting. The individual may or may not have pneumonia or acute respiratory distress syndrome (ARDS).

In specific embodiments, vimentin or a functionally active fragment or derivative thereof is delivered to the individual intravenously, intradermally, transdermally, intrathecally, intraarterially, intraperitoneally, intranasally, intravaginally, intrarectally, topically, intramuscularly, subcutaneously, mucosally, orally, topically, locally, by inhalation, by injection, by infusion, via catheter, and/or via lavage, and the vimentin or a functionally active fragment or derivative thereof may be delivered to the individual once a day, more than once a day, more than once a week, more than once a month, or more than once a year. The vimentin or a functionally active fragment or derivative thereof may be delivered to the individual once or multiple times, and in any case the vimentin or a functionally active fragment or derivative thereof may be provided to an individual by constant infusion. Compositions of the disclosure may further include a pharmaceutically acceptable carrier.

In a further embodiment, provided is a system for treating or preventing coronavirus infection in an individual that comprises one or more substrates having vimentin or a functionally active fragment or derivative thereof (such as a rod domain) bound thereto. Upon exposure of the blood from a coronavirus-infected individual, the viruses in the blood will bind to the substrate, thereby removing the viruses from the blood. In specific embodiments, the system comprises a substrate having vimentin or a functionally active fragment or derivative thereof (such as a rod domain) bound thereto where the substrate is a membrane, tube, or other vessel or container that is coated with vimentin or a functionally active fragment or derivative thereof (such as a rod domain) bound thereto. Methods of use of the system comprise contacting the blood of an individual with a substrate or surface external to the individual which is coated with a composition comprising the vimentin or functionally active fragments or variants thereof disclosed herein. The system can prevent, treat, reduce in severity, and/or delay the onset of coronavirus infection. The vimentin (or fragment or derivative)-bound composition of the system can bind a coronavirus spike protein, in specific cases. The blood of the individual can be returned to the individual after being contacted with the substrate or surface coated with the composition. In some cases, the system is used for an individual that is already infected with coronavirus but use of the system prevents development of one or more symptoms for disease associated with the coronavirus, such as COVID-19, for example. In a specific example, an individual that tests positive for having coronavirus but that is presently asymptomatic is subject to the system to prevent the individual from developing one or more symptoms associated with the coronavirus. In one example, an individual that has one or more symptoms associated with coronavirus is subject to the system to prevent development of one or more additional symptoms and/or to prevent worsening of one or more symptoms, including preventing the onset of pneumonia or ARDS, for example. In some cases, the system further includes an effective amount of a second therapy for the coronavirus infection, which is administered to the individual. In some cases, the system further comprises a test for the coronavirus infection.

In a further embodiment, there is a kit for treating or preventing coronavirus infection in an individual, comprising a composition comprising the vimentin or functionally active fragments or variants thereof disclosed herein and a second therapy for coronavirus infection or prevention, said composition and second therapy housed in one or more suitable containers. The composition can further comprise a pharmaceutically acceptable carrier.

It is specifically contemplated that any limitation discussed with respect to one embodiment of the invention may apply to any other embodiment of the disclosure. Furthermore, any composition of the disclosure may be used in any method of the disclosure, and any method of the invention may be used to produce or to utilize any composition of the disclosure. Aspects of an embodiment set forth in the Examples are also embodiments that may be implemented in the context of embodiments discussed elsewhere in a different Example or elsewhere in the application, such as in the Brief Summary, Detailed Description, Claims, and Brief Description of the Drawings.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1 illustrates the use of recombinant human rod domain of vimentin (rhRod) as a molecular decoy to block SARS-CoV-2 binding to host cells.

FIGS. 2A-2B show biolayer interferometry (BLI) with immobilized SARS-CoV-2 on AR2G biosensors. (FIG. 2A) Raw BLI data showing association and dissociation curves of rhRod to immobilized S with (FIG. 2B) the accompanying steady-state analysis to determine the K_{D} between S and rhRod.

FIGS. 3A-3B shows repeat BLI experiments with immobilized SARS-CoV-2 on AR2G biosensors with a separate rhRod lot. (FIG. 3A) Raw BLI data showing association and dissociation curves of rhRod to immobilized S with (FIG. 3B) the accompanying steady-state analysis to determine the K_{D} between S and rhRod.

FIG. 4. Conceptual framework on how rhRod may treat COVID-19 through (1-4) blocking viral adhesion and replication and (A-C) decreasing neutrophilic infiltration into the lungs.

FIGS. 5A-5B. rhRod naturally forms dimers. (FIG. 5A) Coomassie stain of rhRod (Rod) and rhVim (full length vimetin) in both reducing (R) and non-reducing (NR) conditions. rhRod primarily exists in dimer form. (FIG. 5B) Heat-inactivation was performed by placing rhRod in boiling water for 10 minutes.

FIGS. 6A-6F. Intact rhRod, but not full length vimentin, binds to SARS-CoV-2 spike protein. (FIGS. 6A & 6C) Biolayer interferometry showing association and dissociation of (FIG. 6A) rhRod and (FIG. 6C) boiled rhRod onto immobilized spike protein. (FIG. 6B) Steady state analysis and KD determination (500±44 nM) of rhRod to recombinant spike protein. (FIG. 6D) Compared to the rhRod, binding of heat inactivated (boiled) rhRod to spike protein was non-specific. (FIG. 6E) The affinity between rhRod and spike protein was consistent over several lots of protein created. (FIG. 6F) Recombinant full length vimentin (rhVim) did not bind to immobilized spike protein (performed simultaneously with rhRod and duplicated). This suggests that the head and/or tail domains of vimentin inhibit binding to spike protein.

FIGS. 7A-7B. rhRod blocks SARS-CoV-2 spike protein binding to ACE2. Baseline-corrected curve of BLI data showing rhRod blocking (FIG. 7A) spike protein (S1S2) binding to immobilized ACE2 and (FIG. 7B) ACE2 binding to immobilized S1S2 in a dose dependent fashion.

FIGS. 8A-8C. In silico modeling of spike protein, ACE2, and rhRod interactions predict that rhRod blocks spike protein-ACE2 interactions. Using SwarmDock, (FIG. 8A) rhRod is predicted to bind to spike protein at the ACE2 site, congruent with empirical data that increasing concentrations of rhRod blocks spike-ACE2 interactions. Spike protein is predicted to form hydrogen bonds and salt bridges with rhRod in both (FIG. 8B) monomeric and (FIG. 8C) dimeric forms. Note the increased number of H-bonds and salt bridges in the dimeric rhRod as compared to monomeric rhRod.

FIGS. 9A-9B. rhRod decreases SARS-CoV-2 viral replication. SARS-CoV-2 viral titers as determined by plaque assay on Vero E6 cells. (FIG. 9A) A single dose of rhRod decreases viral replication by 72 hours post infection (HPI). (FIG. 9B) Daily administration of rhRod (1000-4000 nM) decreases viral replication by >100-fold starting at 48 hours post infection. As a secondary control, heat inactivated rhRod (4000 nM; ΔrhRod) did not have any effect on viral replication. Shaded areas are the 95% CI of the slopes. #denotes the rhRod concentrations with negative slopes.

### DETAILED DESCRIPTION

### I. Exemplary Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined. The terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. The term “about” is used according to its plain and ordinary meaning in the area of cell and molecular biology to indicate that a value includes the standard deviation of error for the device or method being employed to determine the value. The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

Recitation of ranges of values herein are merely intended to serve as a shorthand method of referring individually to each separate value falling within the range, unless otherwise indicated herein, and each separate value is incorporated into the specification as if it were individually recited herein.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment.

Furthermore, the particular features, structures, or characteristics may be combined in any suitable manner in one or more embodiments. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The terms “subject,” “individual,” and “patient” and “individual” may be used interchangeably and typically comprise a mammal, in certain embodiments a human or a non-human primate. While the compositions and methods are described herein with respect to use in humans, they are also suitable for animal, e.g., veterinary use. Thus certain illustrative organisms include, but are not limited to humans, non-human primates, canines, equines, felines, porcines, ungulates, lagomorphs, and the like. Accordingly, certain embodiments contemplate the compositions and methods described herein for use with domesticated mammals (e.g, canine, feline, equine), laboratory mammals (e.g, mouse, rat, rabbit, hamster, guinea pig), and agricultural mammals (e.g, equine, bovine, porcine, ovine), and the like. The term “subject” does not require one to have any particular status with respect to a hospital, clinic, or research facility (e.g, as an admitted patient, a study participant, or the like). Accordingly, in various embodiments, the subject can be a human (e.g, adult male, adult female, adolescent male, adolescent female, male child, female child) under the care of a physician or other health worker in a hospital, psychiatric care facility, as an outpatient, or other, clinical context. In certain embodiments, the subject may not be under the care or prescription of a physician, or other, health worker. In certain embodiments the subject may not be under the care a physician or health worker and, in certain embodiments, may self-prescribe and/or self-administer the compounds described herein. The subject may be of any gender, race, or age.

As used herein, the phrase “subject in need thereof” or “individual in need thereof” refers to a subject or individual, as described infra, that suffers or is at a risk of suffering (e.g, pre-disposed such as genetically pre-disposed, or subjected to environmental conditions that pre-dispose, etc.) from the diseases or conditions listed herein (e.g, coronavirus infection).

As used herein, the term “therapeutically effective amount” is synonymous with “effective amount”, “therapeutically effective dose”, and/or “effective dose” refers to an amount of an agent sufficient to ameliorate at least one symptom, behavior or event, associated with a pathological, abnormal or otherwise undesirable condition, or an amount sufficient to prevent or lessen the probability that such a condition will occur or re-occur, or an amount sufficient to delay worsening of such a condition. Effective amount can also mean the amount of a compound, material, or composition comprising a compound of the present disclosure that is effective for producing some desired effect, e.g., preventing binding of coronavirus to cell surface adhesion molecules. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly. Further, one of skill in the art recognizes that an amount may be considered effective even if the medical condition is not totally eradicated but improved partially. For example, the medical condition may be halted or reduced or its onset delayed, a side effect from the medical condition may be partially reduced or completed eliminated, and so forth.

As used herein, the terms “treatment,” “treat,” or “treating” refers to intervention in an attempt to alter the natural course of the individual or cell being treated, and may be performed either for prophylaxis or during the course of pathology of a disease or condition such as for example coronavirus infection comprising pneumonia, organ failure, blood clots, respiratory failure, heart conditions such as cardiomyopathies, acute kidney injury, further viral and bacterial infections, or a combination thereof. Treatment may serve to accomplish one or more of various desired outcomes, including, for example, preventing occurrence or recurrence of disease, alleviation of symptoms, and diminishment of any direct or indirect pathological consequences of the disease, lowering the rate of disease progression, amelioration or palliation of the disease state, remission or improved prognosis, and/or producing some desired effect, e.g., preventing binding of coronavirus to cell surface adhesion molecules. The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel.

### II. General Embodiments

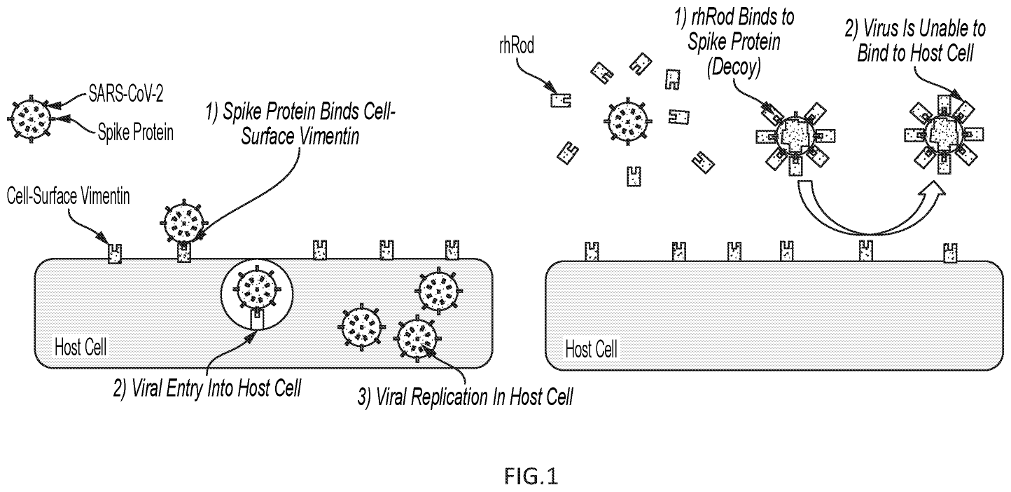

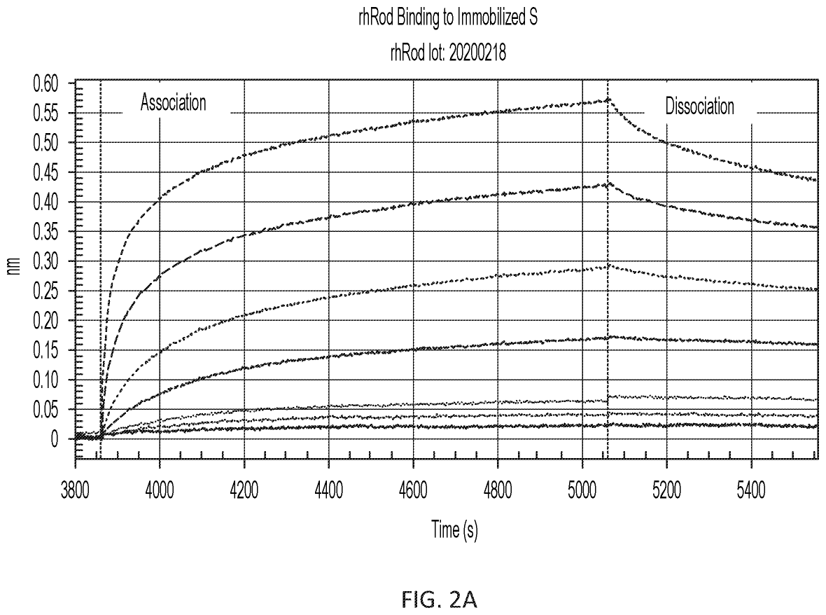

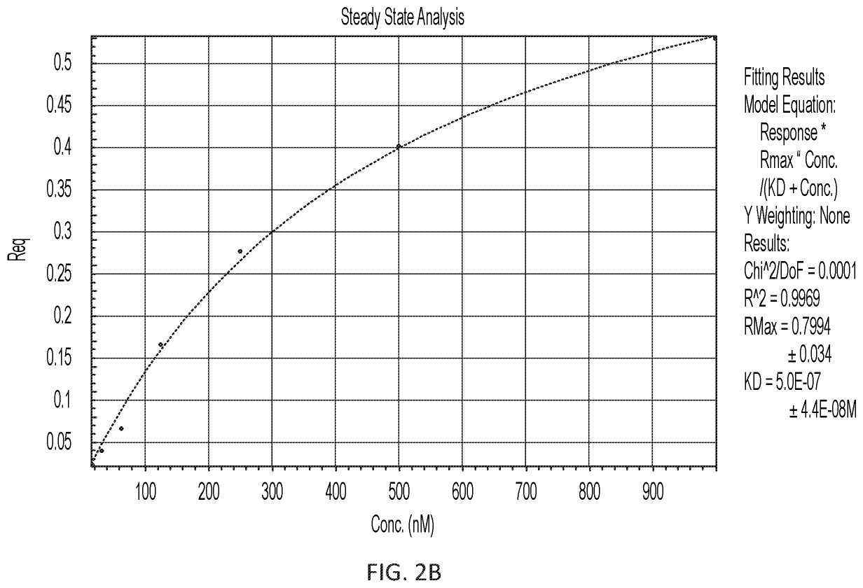

In particular embodiments, the disclosure concerns compositions, methods, and systems that modulate coronavirus infection in an individual, including viral interaction with cells and/or tissues. One way to block viral interaction with cells is through inhibiting the binding of the virus to cell surface receptors. Vimentin is an intracellular protein that is important for maintaining the cytoskeleton as well as intracellular transport. It is primarily located within mesenchymal cells but has also been reported on the surface of cells as well as secreted into the plasma. It was previously shown that rhVim binds selectively to P-selectin to block P-selectin-P-selectin glycoprotein ligand-1 interactions (5), and a recombinant form of the rod domain for potential therapeutic use was previously developed. As described herein, soluble recombinant human rod domain of vimentin (rhRod) (as one example of a fragment of vimentin) can also bind viral spike protein to prevent it from interacting with and binding to cell surface vimentin or other cell surface receptors like ACE2 (FIG. 1), as an example of a mechanism. Further, as shown in FIGS. 2 & 3, robust binding can occur between S and rhRod.

In certain aspects, coronavirus infections are related to SARS or respiratory infections, including SARS-CoV and SARS-CoV-2. SARS or respiratory infections are associated with a variety of complications or medical conditions, including at least pneumonia, organ failure, blood clots, respiratory failure, heart conditions such as cardiomyopathies, acute kidney injury, further viral and bacterial infections, or a combination thereof. In particular embodiments of the disclosure, there is a decrease morbidity and mortality from pneumonia, organ failure, blood clots, respiratory failure, heart conditions such as cardiomyopathies, acute kidney injury, and/or further viral and bacterial infections by modulating coronavirus infection, especially because there are no effective therapies to prevent or treat this disease. Without wishing to be bound by theory, rhRod can inhibit coronavirus infection and the resulting complications by blocking viral infiltration into the lungs. Specifically, rhRod can bind coronavirus spike proteins to prevent coronavirus interacting with cell surface receptors. Thus, by modulating viral infection, one can reduce morbidity and mortality from coronavirus, resulting SARS or respiratory infections, and complications or medical conditions including pneumonia, organ failure, respiratory failure, blood clots, heart conditions such as cardiomyopathies, acute kidney injury, and/or further viral and bacterial infections.

### III. Coronavirus & SARS-CoV-2

Coronaviruses make up a large family of viruses that can infect birds and mammals, including humans, according to the World Health Organization (WHO). These viruses have been responsible for several outbreaks around the world, including the severe acute respiratory syndrome (SARS) pandemic of 2002-2003 and the Middle East respiratory syndrome (MERS) outbreak in South Korea in 2015. Most recently, a novel coronavirus (SARS-CoV-2, also known as SARS-associated coronavirus 2 or COVID-19) triggered an outbreak in China in December 2019.

Coronaviruses constitute the subfamily Orthocoronavirinae, in the family Coronaviridae, order Nidovirales, and realm Riboviria. They are enveloped viruses having a positive-sense single-stranded RNA genome and a nucleocapsid having helical symmetry. The genome size of coronaviruses ranges from about 26-32 kilobases. They have characteristic club-shaped spikes that project from their surface, which in electron micrographs create an image reminiscent of the solar corona, from which their name derives. The average diameter of the virus particles is around 120 nm (0.12 μm). The diameter of the envelope is ˜80 nm (0.08 μm) and the spikes are ˜20 nm (0.02 μm) long. Beneath a coronavirus's spiked exterior is a round core shrouded in a viral envelope. The core contains genetic material that the virus can inject into vulnerable cells to infect them.

The viral envelope consists of a lipid bilayer where the membrane (M), envelope (E), and spike (S) structural proteins are anchored. Inside the envelope, there is the nucleocapsid of helical symmetry which is formed from multiple copies of the nucleocapsid (N) protein, which are bound to the positive-sense single-stranded RNA genome in a continuous beads-on-a-string type conformation. The genome size of coronaviruses ranges from approximately 26 to 32 kilobases, one of the largest among RNA viruses. The genome organization for a coronavirus is 5′-leader-UTR-replicase/transcriptase-spike (S)-envelope (E)-membrane (M)-nucleocapsid (N)-3′UTR-poly (A) tail. The open reading frames 1a and 1b, which occupy the first two-thirds of the genome, encode the replicase/transcriptase polyprotein. The replicase/transcriptase polyprotein self cleaves to form nonstructural proteins. The later reading frames encode the four major structural proteins: spike, envelope, membrane, and nucleocapsid. Interspersed between these reading frames are the reading frames for the accessory proteins. The number of accessory proteins and their function is unique depending on the specific coronavirus.

The lipid bilayer envelope, membrane proteins, and nucleocapsid protect the virus when it is outside the host cell. The spike proteins extend from within the core to the viral surface and allow the virus to recognize and bind specific cells in the body. When the spike engages a receptor on a host cell, a cascade is triggered, resulting in the merger of the virus with the cell which allows the virus to release its genetic material and overtake the cell's processes to produce new viruses.

Several coronaviruses utilize animals as their primary hosts and have evolved to infect humans, too. Precursors to both SARS and MERS coronaviruses appear in bats. The SARS virus jumped from bats to civets (small, nocturnal mammals) on its way into people, while MERS infected camels before spreading to humans. Evidence suggests that the novel coronavirus also jumped from bats to humans after passing through an intermediate carrier, although scientists have not yet identified the infectious middleman creature. Human coronaviruses were first identified in the mid-1960s. There are four main sub-groupings of coronaviruses, known as alpha, beta, gamma, and delta, and seven coronaviruses that can infect people. The four most common coronaviruses did not jump from animals to humans but rather utilize humans as their natural hosts; these include: 229E (alpha coronavirus); NL63 (alpha coronavirus); OC43 (beta coronavirus); HKU1 (beta coronavirus). Three other human coronaviruses are: MERS-CoV (the beta coronavirus that causes Middle East Respiratory Syndrome, or MERS); SARS-CoV (the beta coronavirus that causes severe acute respiratory syndrome, or SARS); and SARS-CoV-2 (the novel coronavirus that causes coronavirus disease 2019, or COVID-19).

Data has shown that the viruses can spread from person to person among those in close contact (within about 6 feet, or 2 meters). The viruses spread by respiratory droplets released when someone with the virus coughs, sneezes, or talks. These droplets can be inhaled or land in the mouth or nose of a person nearby. They can also spread if a person touches a surface with a virus on it and then touches his or her mouth, nose, or eyes.

Infection begins when the viral spike (S) glycoprotein attaches to its complementary host cell receptor. After attachment, a protease of the host cell cleaves and activates the receptor-attached spike protein. Depending on the host cell protease available, cleavage and activation allows the virus to enter the host cell by endocytosis or direct fusion of the viral envelop with the host membrane. On entry into the host cell, the virus particle is uncoated, and its genome enters the cell cytoplasm. The coronavirus RNA genome has a 5′ methylated cap and a 3′ polyadenylated tail, which allows the RNA to attach to the host cell's ribosome for translation. The host ribosome translates the initial overlapping open reading frame of the virus genome and forms a long polyprotein. The polyprotein has its own proteases which cleave the polyprotein into multiple nonstructural proteins.

Viral entry is followed by replication of the virus. A number of the nonstructural proteins coalesce to form a multi-protein replicase-transcriptase complex (RTC). The main replicase-transcriptase protein is the RNA-dependent RNA polymerase (RdRp). It is directly involved in the replication and transcription of RNA from an RNA strand. The other nonstructural proteins in the complex assist in the replication and transcription process. The exoribonuclease nonstructural protein, for instance, provides extra fidelity to replication by providing a proofreading function which the RNA-dependent RNA polymerase lacks. One of the main functions of the complex is to replicate the viral genome. RdRp directly mediates the synthesis of negative-sense genomic RNA from the positive-sense genomic RNA. This is followed by the replication of positive-sense genomic RNA from the negative-sense genomic RNA. The other important function of the complex is to transcribe the viral genome. RdRp directly mediates the synthesis of negative-sense subgenomic RNA molecules from the positive-sense genomic RNA. This is followed by the transcription of these negative-sense subgenomic RNA molecules to their corresponding positive-sense mRNAs.

The replicated positive-sense genomic RNA becomes the genome of the progeny viruses. The mRNAs are gene transcripts of the last third of the virus genome after the initial overlapping reading frame. These mRNAs are translated by the host's ribosomes into the structural proteins and a number of accessory proteins. RNA translation occurs inside the endoplasmic reticulum. The viral structural proteins S, E, and M move along the secretory pathway into the Golgi intermediate compartment. There, the M proteins direct most protein-protein interactions required for assembly of viruses following its binding to the nucleocapsid. Progeny viruses are then released from the host cell by exocytosis through secretory vesicles.

The interaction of the coronavirus spike protein with its complement host cell receptor is central in determining the tissue tropism, infectivity, and species range of the virus. Coronaviruses mainly target epithelial cell receptors. They are transmitted from one host to another host, depending on the coronavirus species, by either an aerosol, fomite, or fecal-oral route. Human coronaviruses infect the epithelial cells of the respiratory tract, while animal coronaviruses generally infect the epithelial cells of the digestive tract. SARS coronavirus infects, for example, via an aerosol route, human epithelial cells of the lungs by binding to the angiotensin-converting enzyme 2 (ACE2) receptor.

The WHO has reported that the two groups most at risk of experiencing severe illness due to a coronavirus infection are adults aged 65 years or older and people who have other underlying health conditions including chronic lung disease, serious heart conditions, severe obesity, a compromised immune system, or diabetes. In humans, coronaviruses typically cause a respiratory infection with mild to severe flu-like symptoms, but the exact symptoms vary depending on the type of coronavirus. The four common human coronaviruses can cause people to develop a runny nose, headache, cough, sore throat and fever, according to the CDC. In a subset of individuals, including those with cardiopulmonary disease or a weakened immune system, the viral infection can progress to a more severe lower-respiratory infection such as pneumonia or bronchitis. In comparison, severe MERS and SARS infections often progress to pneumonia. Other symptoms of MERS include fever, coughing, and shortness of breath, while SARS can cause fever, chills and body aches.

SARS-CoV-2 causes symptoms similar to those of other coronaviruses, triggering fever, cough, and shortness of breath in most patients. Rarer symptoms include dizziness, tiredness, aches, chills, sore throat, loss of smell, loss of taste, headache, nausea, vomiting, and diarrhea. Emergency signs or symptoms can include trouble breathing, persistent chest pain or pressure, new confusion, and/or blue lips or face. Complications of SARS-CoV-2 infections can include pneumonia, organ failure, respiratory failure, blood clots, heart conditions such as cardiomyopathies, acute kidney injury, and/or further viral and bacterial infections.

The present disclosure encompasses treatment or prevention of infection of any virus in the Coronaviridae family. In certain embodiments, the disclosure encompasses treatment or prevention of infection of any virus in the subfamily Coronavirinae and including the four genera, Alpha-, Beta-, Gamma-, and Deltacoronavirus. In specific embodiments, the disclosure encompasses treatment or prevention of infection of any virus in the genus of Betacoronavirus, including the subgenus Sarbecovirus and including the species of severe acute respiratory syndrome-related coronavirus. In specific embodiments, the disclosure encompasses treatment or prevention of infection of any virus in the species of severe acute respiratory syndrome-related coronavirus, including the strains severe acute respiratory syndrome coronavirus (SARS-CoV) and severe acute respiratory syndrome coronavirus 2 (SARS-CoV-2, the virus that causes COVID-19). The disclosure encompasses treatment or prevention of infection any isolate, strain, type (including Type A, Type B and Type C; Forster et al., 2020, PNAS, https://doi.org/10.1073/pnas.2004999117), cluster, or sub-cluster of the species of severe acute respiratory syndrome-related coronavirus, including at least SARS-CoV-2. In specific embodiments, the virus being treated with methods and compositions of the disclosure is not SARS-CoV and is not MERS-CoV. In specific embodiments, the virus being treated with methods and compositions of the disclosure is SARS-CoV or is MERS-CoV. In specific embodiments, the virus has a genome length between about 29000 to about 30000, between about 29100 and 29900, between about 29200 and 29900, between about 29300 and 29900, between about 29400 and 29900, between about 29500 and 29900, between about 29600 and 29900, between about 29700 and 29900, between about 29800 and 29900, or between about 29780 and 29900 base pairs in length.

Examples of specific SARS-CoV-2 viruses include the following listed in the NCBI GenBank® Database, and these GenBank® Accession sequences are incorporated by reference herein in their entirety: (a) LC534419 and LC534418 and LC528233 and LC529905 (examples of different strains from Japan); (b) MT281577 and MT226610 and NC_045512 and MN996531 and MN908947 (examples of different strains from China); (c) MT281530 (Iran); (d) MT126808 (Brazil); (e) MT020781 (Finland); (f) MT093571 (Sweden); (g) MT263074 (Peru); (h) MT292582 and MT292581 and MT292580 and MT292579 (examples of different strains from Spain); (i) examples from the United States, such as MT276331 (TX); MT276330 (FL); MT276328 (OR) MT276327 (GA); MT276325 (WA); MT276324 (CA); MT276323 (RI); MT188341 (MN); and (j) MT276598 (Israel). In particular embodiments, the disclosure encompasses treatment or prevention of infection of any of these or similar viruses, including viruses whose genome has at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. In particular embodiments, the disclosure encompasses treatment or prevention of infection of any of these or similar viruses, including viruses whose genome has its entire sequence that is greater than 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. As one specific example, the present disclosure includes methods of treatment or prevention of infection of a virus having a genome sequence as represented by GenBank® Accession No. NC_045512 (origin Wuhan, China) and any virus having a genome sequence with at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to that sequence. Infection with any strain of SARS-CoV-2 may be treated or prevented, including at least B.1.526, B.1.526.1, B.1.525, B.1.1.7, B.1.351, B.1.427, B.1.429, B.1.617, P.1, and P.2.

Doctors can test for coronavirus infections by analyzing respiratory specimens and serum isolated from blood. The CDC has developed an equivalent diagnostic test for SARS-CoV-2, but its accuracy and specificity for the virus are still being verified. As of yet, there are no available treatments for or vaccines to prevent any human coronavirus. Those who catch a common coronavirus usually recover on their own. Similar regimens are used to relieve the symptoms of more severe coronavirus infections.

### IV. Vimentin and Related Compositions

In embodiments of the disclosure, there are compositions that encompass part or all of vimentin. Although the vimentin may be from any mammal, including mice, rat, chimpanzee, dog, cat, cow, pig, and so forth, but in specific embodiments the vimentin is from human. This is because the vimentin sequence homology between different animals is high. Although the vimentin composition may be isolated from a mammal (and may or may not be modulated thereafter), in specific embodiments the vimentin composition is synthetically generated, such as by recombinant means. In specific embodiments, the vimentin is soluble, as opposed to being cell surface vimentin or intracellular vimentin. The vimentin may be synthetically produced, in certain cases, or may be produced in bacteria, yeast, insect cells, mammalian cells, etc. The vimentin may be soluble recombinant human vimentin (srhVim) or naturally occurring vimentin. The vimentin may be soluble recombinant human rod domain of vimentin (rhRod). In particular cases, the vimentin is administered exogenously (and therefore external to cells). In specific embodiments the vimentin is recombinant. In some cases, the vimentin is in monomer, dimer, or tetramer form. In certain cases the vimentin is glycosylated, whereas in other cases it is not glycosylated.

In particular embodiments, the vimentin composition comprises the entirety of SEQ ID NO:1, although in other embodiments the vimentin composition may comprise a functionally equivalent variant of SEQ ID NO:1. The term “functionally equivalent variant” refers to a polynucleotide or polypeptide sequence that has been modified by substitution, insertion or deletion of one or more (e.g. 1, 2, 3, 4, 5, 6, 7, 8, 9 10) nucleotides or one or more (e.g. 1, 2, 3, 4, 5, 6, 7, 8, 9, 10) amino acids, respectively, but that has substantially the same or better activity as the reference sequence. Function of a polypeptide may be assessed experimentally, for example, by determining activity in an in vitro or in vivo experiment. Whether or not a given polypeptide or polynucleotide is “functional” may be determined by first selecting an appropriate function to assess. The functionality of vimentin and/or a variant or derivative may be assessed as the following: (1) in vitro testing to bind to cell surface receptors, such as but not limited to P-selectin, E-selectin, PSGL-1, etc.; (2) in vitro testing of srhVim or its components on WBC rolling and adhesion to other cell types, such as but not limited to endothelial cells, platelets, cell adhesion molecules; (3) in vivo testing of vimentin on preventing or treating coronavirus infection. In some cases, a functional molecule may be one that exhibits the desired function to a statistically significant degree (e.g. p<0.05; <0.01; <0.001). As a reference sequence, a vimentin polypeptide sequence is in the National Center for Biotechnology Information's GenBank® database at Accession Number NP_003371.

NP_003371
(SEQ ID NO: 1)

| | |
|---|---|
| 1 | mstrsvssss yrrmfggpgt asrpsssrsy vttstrtysl |
| | gsalrpstsr slyasspggv |
| 61 | yatrssavrl rssvpgvrll qdsvdfslad aintefkntr |
| | tnekvelqel ndrfanyidk |
| 121 | vrfleqqnki llaeleqlkg qgksrlgdly eeemrelrrq |
| | vdqltndkar veverdnlae |
| 181 | dimrlreklq eemlqreeae ntlqsfrqdv dnaslarldl |
| | erkveslqee iaflkklhee |
| 241 | eigelqaqiq eqhvqidvdv skpdltaalr dvrqqyesva |
| | aknlqeaeew ykskfadlse |
| 301 | aanrnndalr qakqesteyr rqvqsltcev dalkgtnesl |
| | erqmremeen faveaanyqd |
| 361 | tigrlqdeiq nmkeemarhl reyqdllnvk maldieiaty |
| | rkllegeesr islplpnfss |
| 421 | Inlretnlds lplvdthskr tlliktvetr dgqvinetsq |
| | hhddle |

In some embodiments, a fragment of vimentin is utilized that comprises one or more specific domains of vimentin. In specific cases, the vimentin fragment utilizes the rod, tail, and/or head domains. For example, a vimentin composition may comprise, consist of, or consist essentially of the rod domain, the tail domain, or the head domain, or portions thereof.

In specific embodiments, a vimentin composition comprises a fragment of vimentin that is at least 10, 12, 15, 18, 20, 25, 30, 40, 50, 60, 70, 80, 90, 100,110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, or 460 amino acids in length. In some embodiments, a vimentin composition comprises a fragment of vimentin that is no more than 10, 12, 15, 20, 25, 30, 40, 50, 60, 70, 80, 90, 100,110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, or 460 amino acids in length. As an alternative to, or in addition to, the fragment having a certain length, the fragment may comprise sequence that is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to SEQ ID NO:1. In some cases, sequential fragments of variable lengths that may extend between domains, e.g., include aa70-120, aa390-420, etc. of SEQ ID NO:1 (but not limited to these regions).

In one embodiment, there is provided an isolated human vimentin polypeptide fragment comprising at least a functional portion of vimentin (SEQ ID NO:1), or a functionally equivalent fragment or derivative thereof. In some embodiments, the polypeptide comprises at least 10, 11, 12, 13, 14, 15, 16, 17, 18, 19, 20, 21, 22, 23, 24, 25, 26, 27, 28, 29, 30, 31, 37, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, or more contiguous amino acids of vimentin (SEQ ID NO: 1). In some embodiments, the functional derivative comprises 1, 2, 3, 4, or 5 amino acid differences, such as conservative amino acid modifications, compared to SEQ ID NO:1. The vimentin fragment may include an N-terminal and/or C-terminal truncation of SEQ ID NO:1, such as of 1, 2, 3, 4, 5, 6, 7, 8, 9, 10, 15, 20, 25, or more amino acids truncated from the N-terminal and/or C-terminal of SEQ ID NO:1.

In some embodiments, any vimentin polypeptide fragment is less than 425, 400, 375, 350, 325, 300, 275, 250, 200, 100, 90, 80, 70, 60, 50, 40, 30, 20, or 10 amino acids in length. In some embodiments any vimentin polypeptide fragment is between 10-450, 50-450, 100-400, or 200-300 amino acids in length. In some embodiments the polypeptide fragment is between 10-60, 20-60, 30-60, 40-60 or 50-60 amino acids in length.

In particular embodiments, a vimentin fragment comprises (or consists of or consists essentially of) amino acids 96-407 of SEQ ID NO:1, and the sequence of such a fragment (which is the rod domain, or rhRod) is as follows and is noted as SEQ ID NO:2:

(SEQ ID NO: 2)

FKNTRTNEKV ELQELNDRFA NYIDKVRFLE QQNKILLAEL
EQLKGQGKSR LGDLYEEEMR ELRRQVDQLT NDKARVEVER
DNLAEDIMRL REKLQEEMLQ REEAENTLQS FRQDVDNASL
ARLDLERKVE SLQEEIAFLK KLHEEEIQEL QAQIQEQHVQ
IDVDVSKPDL TAALRDVRQQ YESVAAKNLQ EAEEWYKSKF
ADLSEAANRN NDALRQAKQE STEYRRQVQS LTCEVDALKG
TNESLERQMR EMEENFAVEA ANYQDTIGRL QDEIQNMKEE
MARHLREYQD LLNVKMALDI EIATYRKLLE GE,

wherein a corresponding polynucleotide that encodes it is SEQ ID NO:3:

TTCAAGAACACCCGCACCAACGAGAAGGTGGAGCTGCAGGAGCTGAATGA
CCGCTTCGCCAACTACATCGACAAGGTGCGCTTCCTGGAGCAGCAGAATA
AGATCCTGCTGGCCGAGCTCGAGCAGCTCAAGGGCCAAGGCAAGTCGCGC
CTGGGGGACCTCTACGAGGAGGAGATGCGGGAGCTGCGCCGGCAGGTGGA
CCAGCTAACCAACGACAAAGCCCGCGTCGAGGTGGAGCGCGACAACCTGG
CCGAGGACATCATGCGCCTCCGGGAGAAATTGCAGGAGGAGATGCTTCAG
AGAGAGGAAGCCGAAAACACCCTGCAATCTTTCAGACAGGATGTTGACAA
TGCGTCTCTGGCACGTCTTGACCTTGAACGCAAAGTGGAATCTTTGCAAG
AAGAGATTGCCTTTTTGAAGAAACTCCACGAAGAGGAAATCCAGGAGCTG
CAGGCTCAGATTCAGGAACAGCATGTCCAAATCGATGTGGATGTTTCCAA
GCCTGACCTCACGGCTGCCCTGCGTGACGTACGTCAGCAATATGAAAGTG
TGGCTGCCAAGAACCTGCAGGAGGCAGAAGAATGGTACAAATCCAAGTTT
GCTGACCTCTCTGAGGCTGCCAACCGGAACAATGACGCCCTGCGCCAGGC
AAAGCAGGAGTCCACTGAGTACCGGAGACAGGTGCAGTCCCTCACCTGTG
AAGTGGATGCCCTTAAAGGAACCAATGAGTCCCTGGAACGCCAGATGCGT
GAAATGGAAGAGAACTTTGCCGTTGAAGCTGCTAACTACCAAGACACTAT
TGGCCGCCTGCAGGATGAGATTCAGAATATGAAGGAGGAAATGGCTCGTC
ACCTTCGTGAATACCAAGACCTGCTCAATGTTAAGATGGCCCTTGACATT
GAGATTGCCACCTACAGGAAGCTGCTGGAAGGCGAG.

In some embodiments, a vimentin fragment comprises a fragment of SEQ ID NO:2, wherein the fragment or derivative is at least 70, 75, 80, 85, 90, 91, 92, 93, 94, 95, 96, 97, 98, or 99% identical to SEQ ID NO:2. In some embodiments, the vimentin variant comprises 1, 2, 3, 4, 5, or more variations compared to SEQ ID NO:2. In some embodiments, a fragment of the rod domain is utilized as a therapeutic composition. In such cases, the fragment of the rod domain is a peptide of the rod domain, such as a peptide having a certain number of contiguous amino acids, such as having at least or no more than 8, 9, 10, 11, 12, 13, 14, 15, 16, 17, 18, 19, 20, 21, 22, 23, 24, 25, 26, 27, 28, 29, 30, 31, 32, 33, 34,3 5, 36, 37, 38, 39, 40, 41, 42, 43, 44, 45, 46, 47, 48, 49, or 50 amino acids.

The vimentin head domain is as follows: M F G G P G T A S R P S S S R S Y V T T S T R T Y S L G S A L R P S T S R S L Y A S S P G G V Y A T R S S A V R L R S S V P G V R L L Q D S V D F S L A D A I N T E (SEQ ID NO:4), wherein a corresponding polynucleotide that encodes it is in SEQ ID NO:5:

TCCACCAGGTCCGTGTCCTCGTCCTCCTACCGCAGGATGTTCGGCGGCCC
GGGCACCGCGAGCCGGCCGAGCTCCAGCCGGAGCTACGTGACTACGTCCA
CCCGCACCTACAGCCTGGGCAGCGCGCTGCGCCCCAGCACCAGCCGCAGC
CTCTACGCCTCGTCCCCGGGCGGCGTGTATGCCACGCGCTCCTCTGCCGT
GCGCCTGCGGAGCAGCGTGCCCGGGGTGCGGCTCCTGCAGGACTCGGTGG
ACTTCTCGCTGGCCGACGCCATCAACACCGAG

The vimentin tail domain is as follows: E S R I S L P L P N F S S L N L R E T N L D S L P L V D T H S K R T L L I K T V E T R D G Q V I N E T S Q H H D D L E (SEQ ID NO:6), wherein a corresponding polynucleotide that encodes it is SEQ ID NO:7:

GAGAGCAGGATTTCTCTGCCTCTTCCAAACTTTTCCTCCCTGAACCTGAG
GGAAACTAATCTGGATTCACTCCCTCTGGTTGATACCCACTCAAAAAGGA
CACTTCTGATTAAGACGGTTGAAACTAGAGATGGACAGGTTATCAACGAA
ACTTCTCAGCATCACGATGACCTTGAATAA

In some cases, the vimentin fragment comprises contiguous amino acids within SEQ ID NO:2, SEQ ID NO:4, or SEQ ID NO:6. Such a fragment may comprise 10, 11, 12, 13, 14, 15, 16, 17, 18, 19, 20, 21, 22, 23, 24, 25, 26, 27, 28, 29, 30, 31, 37, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, or 300 amino acids of SEQ ID NO:2, SEQ ID NO:4, or SEQ ID NO:6, respectively. The vimentin fragment may include an N-terminal and/or C-terminal truncation of SEQ ID NO:2, SEQ ID NO:4, or SEQ ID NO:6, such as of 1, 2, 3, 4, 5, 6, 7, 8, 9, 10, 15, 20, 25, or more amino acids truncated from the N-terminal and/or C-terminal of SEQ ID NO:2, SEQ ID NO:4, or SEQ ID NO:6, respectively.

As an alternative to, or in addition to, a vimentin fragment having a certain length, the fragment may comprise sequence that is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to SEQ ID SEQ ID NO:2, SEQ ID NO:4, or SEQ ID NO:6.

In specific embodiments, full-length vimentin or functional fragments thereof may be modified (such as modified rhRod, rh Head+Rod, rh Rod+Tail, and/or rh Head+Tail). In specific embodiments, one can perform the following: (1) change one or more of the amino acids (either through substituting one or more amino acid(s) for another amino acid(s) and/or adding modifications (such as but not limited to—additional side chain components, glycosylation, ubiquitination, sumolytaion, citrullination, etc.) to existing amino acids or both); (2) delete one or more of the amino acids, or (3) add one or more amino acids to the sequence(s) to improve vimentin and vimentin derivatives' activity (including rhRod) against SARS-CoV-2. In cases wherein an amino acid is substituted, it may or may not be a conservative amino acid substitution. In specific embodiments, 1, 2, 3, 4, 5, 6, 7, 8, 9, 10, or more amino acids of SEQ ID NO:1, SEQ ID NO:2, SEQ ID NO:4, or SEQ ID NO:6 are substituted, modified, or deleted.

In particular, embodiments, the vimentin composition or functional fragments or derivatives thereof are formulated as a pharmaceutical composition. Pharmaceutical compositions of the present disclosure comprise an effective amount of one or more vimentin compositions dissolved or dispersed in a pharmaceutically acceptable carrier. The phrases “pharmaceutical” and “pharmacologically acceptable” and used interchangeably herein refer to molecular entities and compositions that do not produce an adverse, allergic or other untoward reaction when administered to an animal, such as, for example, a human, as appropriate, and do not interfere with the therapeutic methods and systems of the disclosure. The preparation of a pharmaceutical composition that contains at least one vimentin composition or additional active ingredient will be known to those of skill in the art in light of the present disclosure, as exemplified by Remington: The Science and Practice of Pharmacy, 21st Ed. Lippincott Williams and Wilkins, 2005, incorporated herein by reference. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The vimentin compositions (herein also including functional fragments or derivatives thereof) may comprise different types of carriers depending on whether it is to be administered in solid, liquid or aerosol form, and whether it needs to be sterile for such routes of administration, such as injection. The vimentin compositions of the present disclosure can be administered intravenously, intradermally, transdermally, intrathecally, intraarterially, intraperitoneally, intranasally, intravaginally, intrarectally, topically, intramuscularly, subcutaneously, mucosally, intratumorally, orally, topically, locally, inhalation (e.g., aerosol or nebulized inhalation), injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The vimentin composition(s) may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present disclosure suitable for administration may be provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in practicing the methods and systems of the present disclosure is appropriate. Examples of carriers or diluents include fats, oils, water, saline solutions, lipids, liposomes, resins, binders, fillers, alcohols, and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art. The vimentin composition may be lyophilized.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may include the use of a pharmaceutical lipid vehicle compositions that incorporates a vimentin composition, one or more lipids, and an aqueous solvent. As used herein, the term “lipid” will be defined to include any of a broad range of substances that is characteristically insoluble in water and extractable with an organic solvent. This broad class of compounds is well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions, methods, and systems of the present disclosure.

One of ordinary skill in the art would be familiar with the range of techniques that can be employed for dispersing a composition in a lipid vehicle. For example, the vimentin composition(s) may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to the subject can be determined by physical and physiological factors such as body weight, severity of condition, the type of disease being treated, previous or concurrent therapeutic interventions, idiopathy of the subject and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% (by weight) of an active compound. In other embodiments, the active compound may comprise between about 2% to about 75% of the weight of the unit, or between about 25% to about 60%, for example, and any range derivable therein. Naturally, the amount of active compound(s) in each therapeutically useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg body weight, about 5 microgram/kg body weight, about 10 microgram/kg body weight, about 50 microgram/kg body weight, about 100 microgram/kg body weight, about 200 microgram/kg body weight, about 350 microgram/kg body weight, about 500 microgram/kg body weight, about 1 milligram/kg body weight, about 5 milligram/kg body weight, about 10 milligram/kg body weight, about 50 milligram/kg body weight, about 100 milligram/kg body weight, about 200 milligram/kg body weight, about 350 milligram/kg body weight, about 500 milligram/kg body weight, to about 1000 mg/kg body weight or more per administration of the active agent, e.g., a vimentin composition according to the present disclosure, and any range derivable therein. In non-limiting examples of a derivable range from the numbers listed herein, a range of about 1 mg/kg body weight to about 100 mg/kg body weight, about 5 microgram/kg body weight to about 500 milligram/kg body weight, etc., of the active agent can be administered, based on the numbers described above.

Alimentary Compositions and Formulations

In particular embodiments of the present disclosure, the vimentin composition is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration, the vimentin compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively, the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10% (by weight), and preferably about 1% to about 2% (by weight).

Parenteral Compositions and Formulations

In further embodiments, the vimentin compositions may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (see, e.g., U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

Miscellaneous Pharmaceutical Compositions and Formulations

In other preferred embodiments of the disclosure, the active compound vimentin composition may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present disclosure may also comprise the use of a “patch”. For example, the patch may supply one or more active substances at a predetermined rate and in a continuous manner over a fixed period of time.

In certain embodiments, the pharmaceutical vimentin compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (see, e.g., Takenaga et al., 1998) and lysophosphatidyl-glycerol compounds (see, e.g., U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in, e.g., U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present disclosure for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### IV. Examples of Systems and Methods of Treatment or Prevention

Embodiments of the disclosure include systems and methods of delivering a therapeutically effective amount of one or more vimentin compositions to an individual in need thereof. In specific embodiments, the individual has or is at risk for having coronavirus infection. The individual may have a condition that has as a symptom and/or a mechanism an increase in binding of viral particles to cell surface receptors, for example. Embodiments of the disclosure include treatment or prevention of any medical condition in which modulation of coronavirus infection would be beneficial. In specific embodiments, an individual is provided a therapeutically effective amount of one or more vimentin compositions for attenuation of coronavirus infection in an individual, including when the individual has dysregulation of physiological processes following coronavirus infection which can lead to SARS or respiratory infections (including COVID-19/SARS-CoV-2 infection in any embodiment encompassed herein) and complications including pneumonia, organ failure, respiratory failure, blood clots, heart conditions such as cardiomyopathies, acute kidney injury, and/or further viral and bacterial infections.

In specific embodiments, the medical condition treated or prevented with vimentin comprises SARS or respiratory infections which can lead to pneumonia, organ failure, blood clots, respiratory failure, heart conditions such as cardiomyopathies, acute kidney injury, viral and bacterial infections, or a combination thereof.

In particular embodiments, coronavirus infection is not treated with compositions, methods, and systems of the disclosure but instead is prevented, reduced in severity, or there is a delay in onset and/or severity, for example. In some cases, the vimentin treats or prevents the medical condition in the individual by inhibiting coronavirus binding to cell surface receptors such as ACE2, for example.

Embodiments of the disclosure include compositions, methods, and systems that prevent the development of SARS or respiratory infection or the progression from SARS or respiratory infection to pneumonia, organ failure, respiratory failure, blood clots, heart conditions such as cardiomyopathies, acute kidney injury, and/or further viral and bacterial infections, as a result of coronavirus infection. In some cases, once an individual appears to be at risk for developing SARS or respiratory infection, pneumonia, organ failure, respiratory failure, blood clots, heart conditions such as cardiomyopathies, acute kidney injury, and/or further viral and bacterial infections, or requiring intubation, an individual is given an effective amount of one or more vimentin compositions as part of their care.

In specific cases, delivery of vimentin to an individual blocks the binding of coronavirus to cell surface receptors which leads to SARS or respiratory infection. Specific embodiments of the disclosure include compositions, methods, and systems in which a vimentin composition targets the spike proteins of coronavirus. In particular, vimentin inhibits viral infection by inhibiting coronavirus binding to cell surface receptors via blocking coronavirus spike protein interactions with the cell surface receptors. In particular cases, the rod domain of vimentin preferentially binds to coronavirus spike proteins for a therapeutic outcome.

Although in some cases the vimentin composition is provided as a sole therapy for the individual, in some cases the individual is provided a second therapy. The second therapy may be of any kind, but in specific cases the second therapy is antibiotics, antivirals, convalescent serum from previously infected-individuals recovered from coronavirus, immune modulators, anticoagulants, fluids, oxygen, a corticosteroid, antibodies, GSnP-6, sialyl Lewis X analog, anti-proliferatives, calcineurin inhibitors, anti-signaling compounds, or a combination thereof. Vimentin compositions may also be a second therapy to attenuate SARS or respiratory infection until the primary process is resolved (e.g., resolution of coronavirus infection)

In particular embodiments, an individual that is at risk for coronavirus infection leading to SARS or respiratory infection or that is known to have coronavirus infection is provided a therapeutically effective amount of one or more vimentin compositions. In some cases, the individual has been diagnosed with coronavirus infection or SARS or respiratory infection, for example. In some cases, the individual is at risk of contracting coronavirus infection which can lead to SARS or respiratory infection. Risk factors for contracting coronavirus infection which can lead to SARS or respiratory infection include advanced age and/or underlying medical conditions including chronic lung disease, serious heart conditions, severe obesity, a compromised immune system, or diabetes, and an individual characterized by one or more of these risk factors may be provided an effective amount of one or more vimentin compositions.

In some embodiments, a medical condition is treated or prevented with vimentin or a vimentin-related composition that is delivered to the individual multiple times, such as once a day, more than once a day, twice a day, once a week, more than once a week, once a month, more than once a month, once a year, or more than once a year. The multiple treatments may or may not have the same formulations and/or routes of administration(s). Any administration may be as a continuous infusion.

The provider skilled in the art of medical care and decision may determine an appropriate end-point for vimentin composition therapy based on the specific disease process and clinical course of the patient or individual.

In specific embodiments, an additional viral therapy or preventative may be provided to the individual in combination with the disclosed treatment. In specific embodiments, the additional viral therapy or preventative is for a Coronaviridae family infection (including SARS-CoV-2) selected from the group consisting of Azithromycin, AC-55541, Apicidin, AZ3451, AZ8838, Bafilomycin A1, CCT 365623, Daunorubicin, E-52862, Entacapone, GB110, H-89, Haloperidol, Indomethacin, JQ1, Loratadine, Merimepodib, Metformin, Midostaurin, Migalastat, Mycophenolic acid, PB28, PD-144418, Ponatinib, Ribavirin, RS-PPCC, Ruxolitinib, RVX-208, S-verapamil, Silmitasertib, TMCB, UCPH-101, Valproic Acid, XL413, ZINC1775962367, ZINC4326719, ZINC4511851, ZINC95559591, 4E2RCat, ABBV-744, Camostat, Captopril, CB5083, Chloramphenicol, Chloroquine (and/or Hydroxychloroquine), CPI-0610, Dabrafenib, DBeQ, dBET6, IHVR-19029, Linezolid, Lisinopril, Minoxidil, ML240, MZ1, Nafamostat, Pevonedistat, PS3061, Rapamycin (Sirolimus), Sanglifehrin A, Sapanisertib (INK128/M1N128), FK-506 (Tacrolimus), Ternatin 4 (DA3), Tigecycline, Tomivosertib (eFT-508), Verdinexor, WDB002, Zotatifin (eFT226), remdesivir, dexamethasone, tocilizumab, and a combination thereof.

### V. Kits

Any of the vimentin or vimentin-related compositions described herein may be part of a kit. The kits may comprise a suitably aliquoted vimentin composition of the present disclosure, and the component(s) of the kits may be packaged either in aqueous media or in lyophilized form. The container of the kits will generally include at least one vial, test tube, flask, bottle, syringe or other container, into which a component may be placed, and preferably, suitably aliquoted. Where there is more than one component in the kit, the kit also will generally contain a second, third or other additional container into which the additional component(s) may be separately placed. However, various combinations of components may be comprised in a vial. The kits of the present disclosure also will typically include a container for holding the vimentin composition and any other reagent containers in close confinement for commercial sale.

When the components of the kit are provided in one and/or more liquid solutions, the liquid solution is an aqueous solution, with a sterile aqueous solution being contemplated. The compositions may also be formulated into a syringeable composition. In which case, the container may itself be a syringe, pipette, and/or other such like apparatus, from which the formulation may be applied to a particular area of the body, injected into an individual, and/or even applied to and/or mixed with the other components of the kit. However, the component(s) of the kit may be provided as dried powder(s). When reagents and/or components are provided as a dry powder, the powder can be reconstituted by the addition of a suitable solvent. It is envisioned that the solvent may also be provided in another container.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Biolayer Interferometry (Bli) with Immobilized Sars-Cov-2 S on Ar2G Biosensors

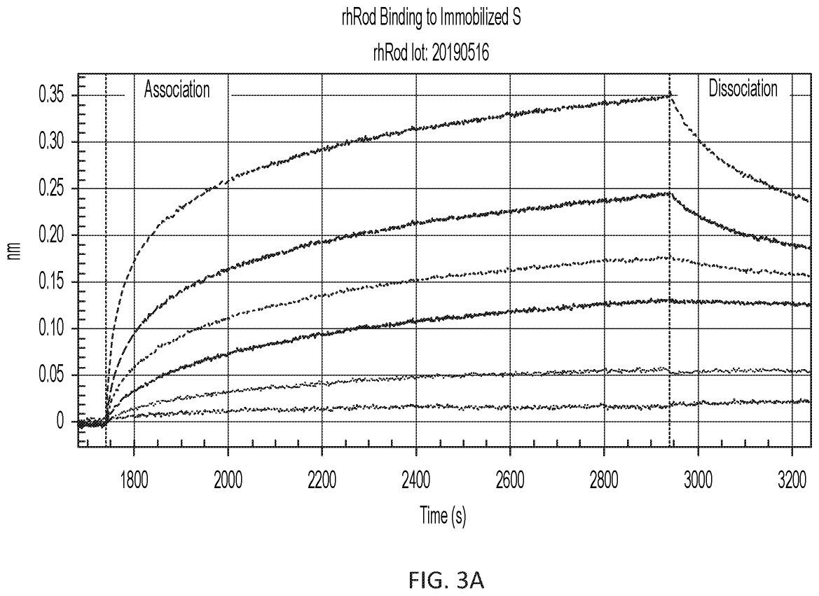

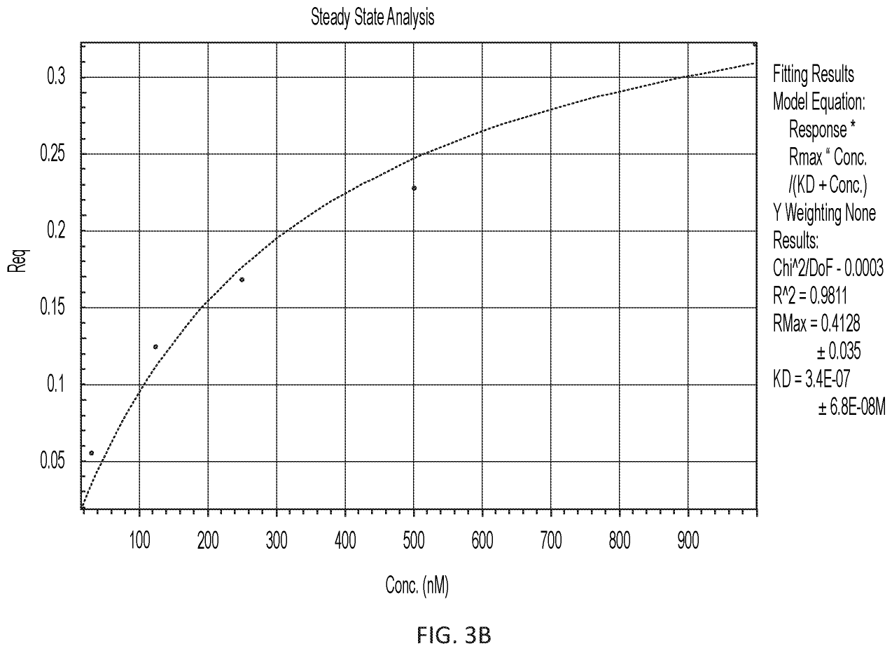

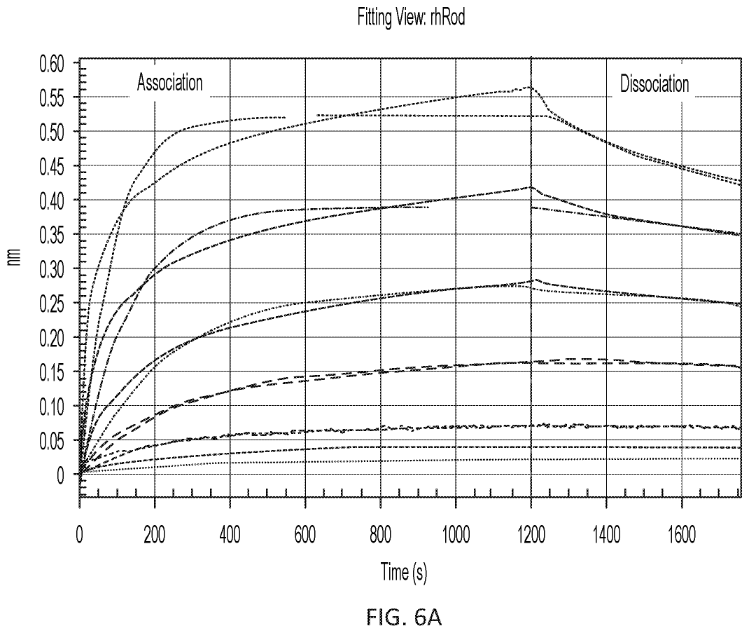

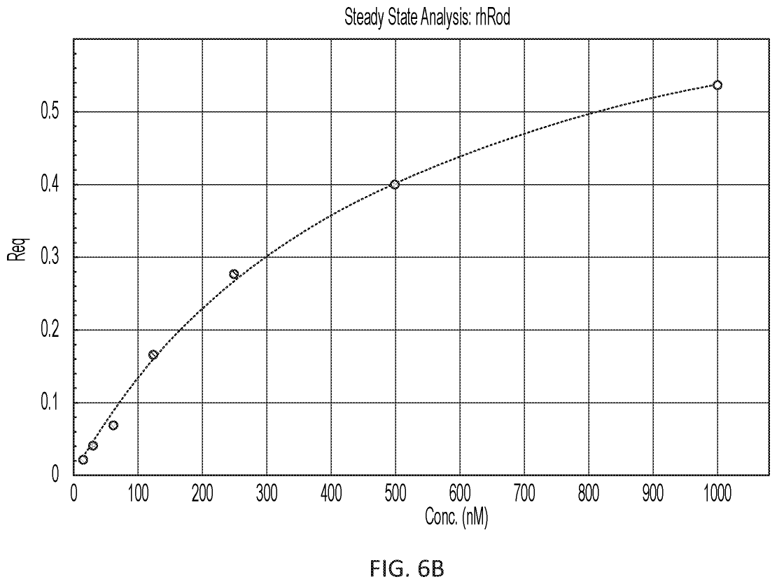

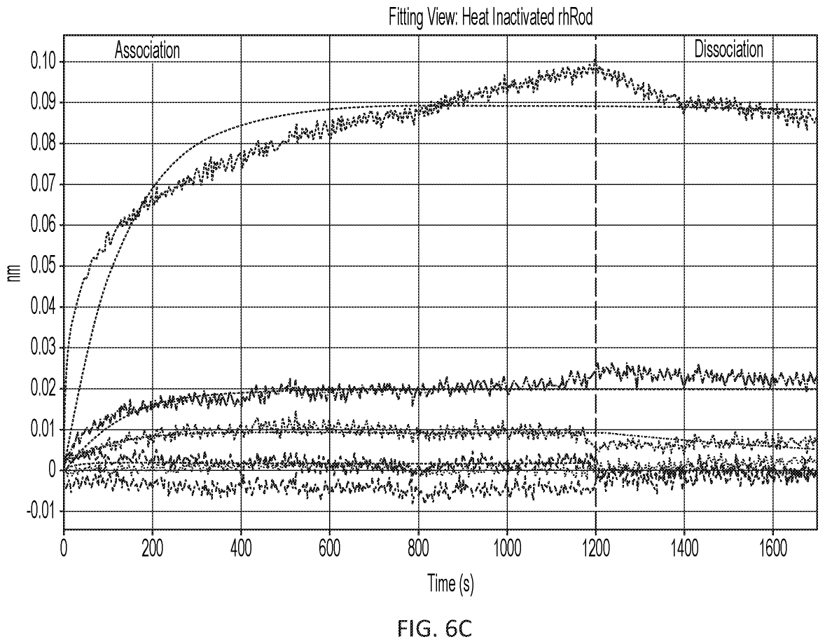

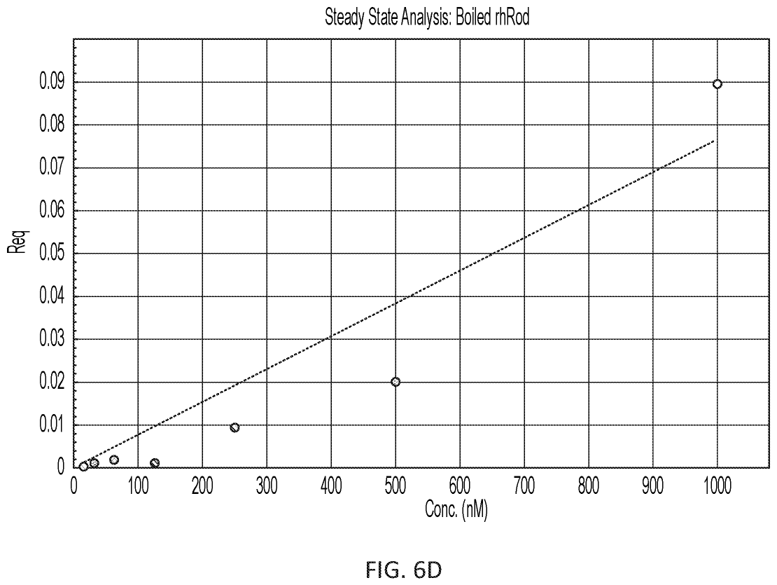

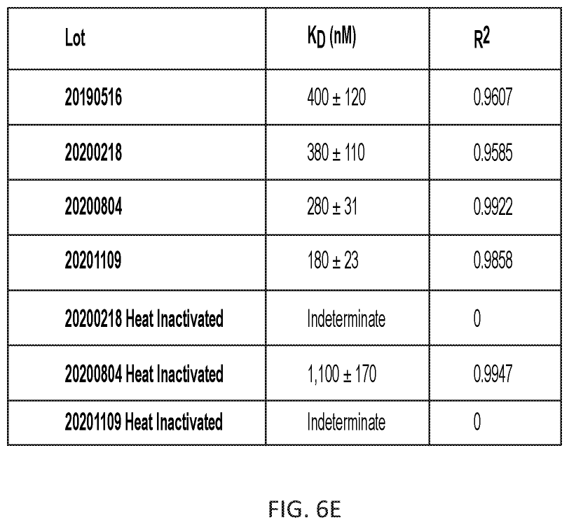

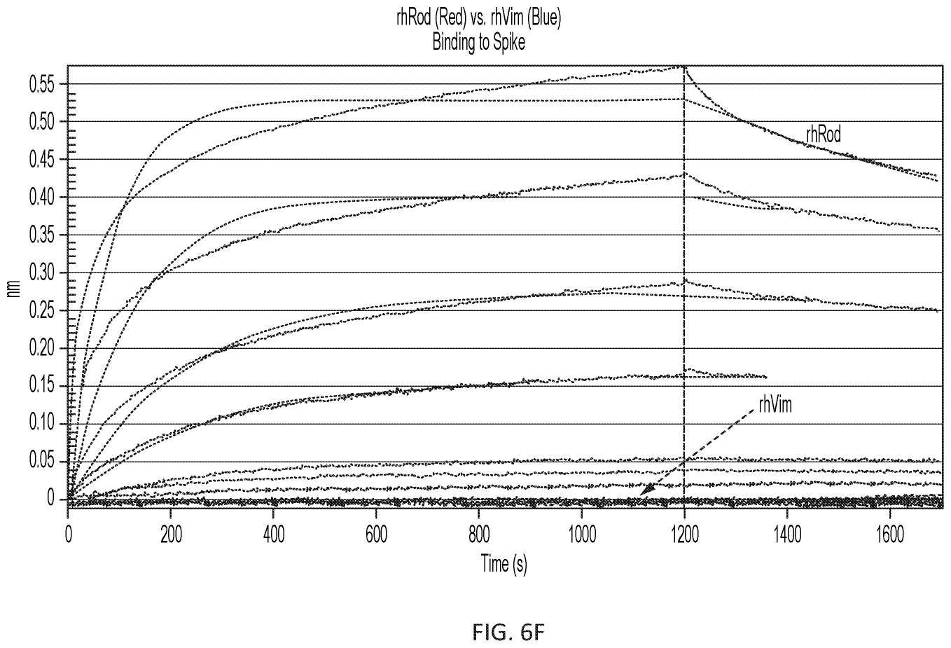

Binding kinetics between immobilized recombinant SARS-CoV-2 spike protein (S) and rhRod was performed using biolayer interferometry (BLI). First, SARS-CoV-2 spike protein was immobilized on amine reactive second generation biosensors. The sensors underwent regeneration (cleaning), and were then used to measure the binding affinity (K_{D}) of immobilized S to rhRod in solution at different concentrations in parallel (0-1,000 nM). Biosensors underwent regeneration between analyses. There was robust binding between S and rhRod (K_{D} 500±44 nM; R₂ 0.9969; R_{Max} 0.7994±0.034; lot: rhRod 20200218; FIG. 2). As shown in FIG. 3, repeat experiments with a separate lot yielded similar results: rhRod (K_{D} 340±68 nM; R₂ 0.9811; R_{Max}0.4128±0.035; lot rhRod 20190516). Also, as shown in FIG. 6E, there is consistent binding among multiple lots of rhRod (spanning over 1 year), and heat inactivation that denatures the protein does not bind (or binds minimally) to SARS-CoV-2 spike protein.

### Example 2

### Determination of the Efficacy of rhRod in Blocking Recombinant Spike Protein Adhesion to Vero E6

Vero E6 cells (ATCC CRL-1586) are cultured onto clear bottom, black-walled 96-well plates to confluence. Recombinant SARS-CoV-2 spike protein (S1+S2 ECD) is added to the wells, in triplicate, in the presence of rhRod, heat denatured rhRod, or vehicle control in a dose dependent fashion (50 μg/mL→0 μg/mL; 100-fold dose based on the K_{D} between S and rhRod). After 1 hour of incubation at 37° C./5% CO₂, cells are washed, primarily labeled with rabbit anti-S1 Ab (Sino Biological), washed, then secondarily labeled with goat anti-rabbit IgG/IR800 (Li-Cor). Finally, wells are imaged for fluorescence intensity on a Li-Cor Odyssey. It is expected that rhRod will decrease recombinant S binding to Vero E6 cells.

### Example 3

### Determination of the Capacity of Immobilized rhRod to Capture Heat Inactivated (HI) SARS-CoV-2

rhRod, heat denatured rhRod, or vehicle control is immobilized on 96-well plates at different concentrations. Plates are blocked with bovine serum albumin and washed. HI SARS-CoV-2 (ATCC VR-1986HK; biosafety level 1) is added to the wells and incubated for 1 hour at room temperature. Following washing, rabbit anti-S1 Ab and goat anti-rabbit IgG/IR800 are used to detect the amount of adhered virus in the wells using the Li-Cor Odyssey. It is expected that immobilized rhRod will capture heat inactivated SARS-CoV-2 in a dose-dependent fashion.

### Example 4

Determination of the Efficacy of rhRod in Blocking Heat Inactivated (HI) SARS-CoV-2 Adhesion to Vero E6

It is unknown whether HI SARS-CoV-2 will bind to Vero E6. However, the use of HI SARS-CoV-2 allows for experiments to be performed outside of BSL3 laboratories, freeing scarce resources (lab space) as well as allowing performance of preliminary tests quickly in the laboratory. Vero E6 cells are cultured onto 24-well plates to confluence. HI SARS-CoV-2 is co-incubated with the cells in the presence of rhRod, heat denatured rhRod, or vehicle control in escalating doses. After washing, rabbit anti-S1 Ab and goat anti-rabbit IgG/IR800 are used to detect the amount of adhered virus in the wells using the Li-Cor Odyssey. It is expected that rhRod will decrease heat-inactivated SARS-CoV-2 binding to Vero E6 cells.

### Example 5

### Determination of the Efficacy of rhRod in Blocking SARS-CoV-2 Adhesion and Replication in Vero E6

Within a BSL3 facility, Vero E6 cells are cultured onto 24-well plates to confluence. Live SARS-CoV-2 (at a 0.1 multiplicity of infection) is incubated in the presence of increasing concentrations of rhRod, heat denatured rhRod, or vehicle control at 37° C./5% CO₂. 72 hours after infection, cells are examined for signs of cytopathology (including detachment, rounding, and degeneration). Additional studies can measure viral genome copy numbers in the presence of rhRod, heat denatured rhRod, or vehicle control as a test for viral replication.

### Example 6

### A System for Treating or Preventing Coronavirus

A system for treating or preventing coronavirus infection in an individual comprises a substrate or surface coated with a composition comprising vimentin or a functionally active fragment or variant thereof. Use of the system may be dialysis-like in nature and comprises contacting the blood of an individual with a substrate or surface coated with a composition comprising vimentin or a functionally active fragment or variant thereof. The system can prevent, reduce in severity, and/or delay the onset of coronavirus infection. In some cases, the system is used for an individual that is already infected with coronavirus but use of the system prevents development of disease associated with the coronavirus, such as COVID-19, for example. In other cases the system is used on an infected individual to prevent development or worsening of one or more symptoms.

In some embodiments,he surface of at least part of the system comprises one or more membranes, tubes, suitable containers, or a combination thereof. In specific embodiments, the fragment of vimentin of the system comprises the N-terminal head domain, C-terminal tail domain, α-helical coiled-coil rod domain, or a combination thereof. The fragment is the N-terminus, the C-terminus, both the N-terminus and C-terminus, or neither of the N-terminus or C-terminus, in specific embodiments. In some cases, the fragment or derivative is at least 70, 75, 80, 85, 90, 91, 92, 93, 94, 95, 96, 97, 98, or 99% identical to SEQ ID NO:1, and the variant is 1, 2, 3, 4, 5, or more variations compared to SEQ ID NO:1, SEQ ID NO:2, SEQ ID NO:4, or SEQ ID NO:6. In some cases, the fragment is SEQ ID NO:2, the fragment or derivative is at least 70, 75, 80, 85, 90, 91, 92, 93, 94, 95, 96, 97, 98, or 99% identical to SEQ ID NO:2, and the variant is 1, 2, 3, 4, 5, or more variations compared to SEQ ID NO:2.

In particular embodiments, when the blood of an individual is passed over or contacted with the surface coated with the composition comprising vimentin or a functionally active fragment or variant thereof, the composition binds a coronavirus spike protein, although in alternative embodiments the vimentin or a functionally active fragment or variant thereof binds a different part of the virus. The blood of the individual is returned to the individual after being contacted with the surface coated with the composition such that the blood becomes depleted or reduced in viral load of coronavirus.

In some cases, the system further comprises an effective amount of a second therapy for the coronavirus infection, which is administered to the individual. The second therapy can be antibiotics, antivirals, convalescent serum, immune modulators, anticoagulants, fluids, oxygen, a corticosteroid, antibodies, GSnP-6, sialyl Lewis X analog, anti-proliferatives, calcineurin inhibitors, anti-signaling compounds, or a combination thereof. The second therapy comprises an anti-SARS-CoV-2 drug. The anti-SARS-CoV-2 drug is selected from the group consisting of Azithromycin, AC-55541, Apicidin, AZ3451, AZ8838, Bafilomycin A1, CCT 365623, Daunorubicin, E-52862, Entacapone, GB110, H-89, Haloperidol, Indomethacin, JQ1, Loratadine, Merimepodib, Metformin, Midostaurin, Migalastat, Mycophenolic acid, PB28, PD-144418, Ponatinib, Ribavirin, RS-PPCC, Ruxolitinib, RVX-208, S-verapamil, Silmitasertib, TMCB, UCPH-101, Valproic Acid, XL413, ZINC1775962367, ZINC4326719, ZINC4511851, ZINC95559591, 4E2RCat, ABBV-744, Camostat, Captopril, CB5083, Chloramphenicol, Chloroquine (and/or Hydroxychloroquine), CPI-0610, Dabrafenib, DBeQ, dBET6, IHVR-19029, Linezolid, Lisinopril, Minoxidil, ML240, MZ1, Nafamostat, Pevonedistat, PS3061, Rapamycin (Sirolimus), Sanglifehrin A, Sapanisertib (INK128/M1N128), FK-506 (Tacrolimus), Ternatin 4 (DA3), Tigecycline, Tomivosertib (eFT-508), Verdinexor, WDB002, Zotatifin (eFT226), and a combination thereof. In some cases, the system further comprises a test for the coronavirus infection.

### Example 7

### Synthetic Vimentin Production

One embodiment of producing synthetic vimentin is as described. Inclusion bodies come from lysed E. coli were solubilized with GHCl. After centrifugation, the solubilized pellet (GHCl-Vim) for 30 min at full speed (20,000 rpm), the supernatant is filtered through 0.45 um and slowly diluted 40× in cold buffer—25 mM Tris-Cl, 1.0 M NaCl, 0.5% Tween-20, pH-7.4 (this buffer is prepared the day before and stored in the cold room). Equilibrate the cold Nickel column with Buffer A (25 mM Tris-Cl, 1.0 M NaCl, 0.5% Tween-20, pH-7.4). If the diluted solution remains crystal clear, then it is suitable for the Nickel column. Wash the column with Buffer A until OD is near baseline. Elute the first peak with 5% Imidazole (Buffer B-500 mM Imidazole, 25 mM Tris-Cl, 1.0 M, NaCl, 0.5% Tween-20, pH-7.4), and continue until OD reach back the baseline. Elute the vimentin protein with 40% Imidazole. The collected peak is then dialyzed against cold 10 mM Na2PO4, pH-8.0 overnight. Filter the solution through 0.2 uM, and check concentration at OD of 280 nM using ε=24,000 cm-1M-1 Mol Weight=55,091 g/moL.

### Example 8

### Vimentin Administration to an Individual

Vimentin, including recombinant vimentin, and/or its functional fragments (such as rhRod) or derivatives thereof, may be utilized in mammalian individuals (such as human subjects or patients), and such administration attenuates the development or progression of acute lung injury (as one example of pathologic inflammation), in particular embodiments. As one example only, an individual that presents to a medical emergency facility (such as an emergency room) with symptoms or risk factors for coronavirus infection (including but not limited to fever, cough, shortness of breath, dizziness, tiredness, aches, chills, sore throat, loss of smell, loss of taste, headache, nausea, vomiting, diarrhea, trouble breathing, persistent chest pain or pressure, new confusion, blue lips or face. or a combination thereof) would be given a dose of recombinant vimentin and/or its fragments or derivatives thereof (such as intravenously and/or via inhalation) in addition to other supportive care (such as antibiotics, antivirals, fluids, oxygen, etc.). The individual's lung function may be monitored using standard clinical variables (such as oxygen saturation, respiratory rate, blood gas measurements, physical examination findings, diagnostic imaging, etc., or a combination thereof). Multiple doses may be utilized if the individual has ongoing worsening of lung function. As the individual improves by means of requiring de-escalation of medical support, the vimentin and/or its fragments or derivatives thereof may be discontinued. Endpoints of successful intervention include stable or improving lung function, such as demonstrated by physical examination and respiratory rate normalization, improving pAO2/FiO₂ ratio (P/F ratio), normalization of pCO₂, preventing need for intubation and mechanical ventilation, (for those mechanically ventilated) decreased number of ventilator days, decreased hospital length of stay, decreased intensive care unit length of stay, or a combination thereof. Doses and/or routes may be varied.

### Example 9

### Recombinant Rod Domain Blocks SARS-CoV-2 Cell Adhesion and Replication

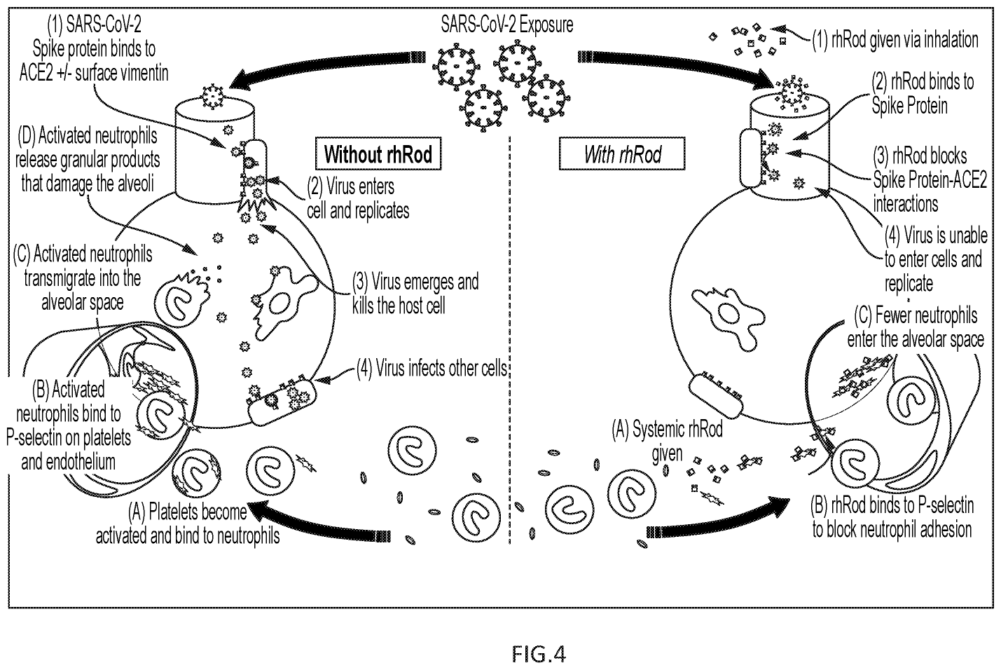

This example concerns using a novel recombinant “mimic” of vimentin (rhRod) to block binding of SARS-CoV-2 spike (S) protein to its cellular receptor, ACE2, and its ability to decrease leukocyte adhesion by blocking P-selectin-P-selectin glycoprotein-1 (PSGL-1) interactions (FIG. 4). Based data where treatment with rhRod led to a decrease in viral load, rhRod in specific embodiments of the disclosure is an effective therapy to treat COVID-19 through at least two distinct mechanisms, in at least some cases: (1) rhRod binds SARS-CoV-2 S protein to prevent attachment to host ACE2, preventing viral entry into the cells and replication, and/or (2) through interaction with P-selectin, leading to a decrease in secondary injury caused by pathologic inflammation by decreasing leukocyte recruitment into the lungs and other organs. This example concerns characterization how rhRod leads to a 3-log decrease in viral load through the use of cell and organoid models and in a transgenic mouse model of COVID-19.

In specific embodiments, there is a step-wise, systematic approach in identifying the biochemical properties of rhRod that confer its ability to bind to SARS-CoV-2 S protein. This leads to development of small, specific peptides that are non-immunogenic. One can optimize in vitro assays using human cell lines to assess the efficacy of rhRod in decreasing SARS-CoV-2 infection and replication. One can characterize the ability of rhRod to block infections by the known mutations of SARS-CoV-2. One can also characterize findings using human airway organoids. Studying organoids in culture is superior to standard monolayers of cells due to the multicellular nature of organoids in three dimensional structures, as opposed to the typical single cell derived monolayers. The use of organoids from primary human airways will more closely replicate the pathology of SARS-CoV-2 in vitro, and, thus, be more informative in characterizing the efficacy of rhRod in blocking adhesion and replication. Finally, one can compare findings in human airway organoids against animal models of SARS-CoV-2 infection to assess the ability of organoids to reduce the need for animal use for drug screening. One can also generate a novel mouse model to assess the role of P-selectin in the development of COVID-19. One can breed P-selectin−/− mice with the K18-hACE2 mice to create P-selectin^{-/-}/K18-hACE2 (and P-selectin^{+/+}/K18-hACE2) mice.

In specific embodiments, there is a recombinant form of the rod domain of human vimentin (rhRod) as a molecular mimic to bind to viral spike protein to prevent the SARS-CoV-2 virus from binding to and entering cells. Use of rhRod to block viral adhesion to and infection of cells will effectively decrease replication as the virus requires cell entry to multiply as well as to mediate the hyperinflammatory response. Additionally, one can utilize rhRod systemically to decrease ongoing pathologic inflammation through blocking P-selectin-PSGL-1 interactions. These dual effects of rhRod may allow for more tailored therapy to treat patients with COVID-19 based on the degree of pathologic inflammation (and requiring systemic rhRod in addition to intranasal or inhalational rhRod). One can measure the efficacy of rhRod in vitro and in vivo. In the animal studies, the outcomes and organ function are measured in addition to the inflammatory and coagulation profiles, since SARS-CoV-2 infection leads to dysregulation of these pathways. Biochemical techniques are used to create and test shorter candidate peptide sequences to block both viral adhesion and inflammation.

### Identification of Epitopes within rhRod and Develop Shorter Peptide Fragments that Block SARS-CoV-2 Adhesion In Vitro and In Vivo

In specific embodiments, there is systematic identification of the binding epitopes on rhRod (base pairs 717-1,652; aa 96-407 on human vimentin; sequence NM_00380.3 [NCBI])) to which spike protein binds using SPOT peptide arrays. Alanine scanning may be performed followed by generation of positional scanning libraries to identify the key residues responsible for the interaction. Through this process, one can identify the biochemical properties through which this interaction occurs. Biolayer interferometry may be used to measure the KD between peptide fragments and spike protein as well as the IC50 of novel peptide fragments in blocking spike-ACE2 binding. Using live SARS-CoV-2 in our BSL3 facility, one can characterize candidate peptides in vitro on cell culture monolayers and human airway organoids to identify the EC50 of each. Finally, top candidates are tested in vivo using K18-hACE2 mice, who are susceptible to SARS-CoV-2 infection.

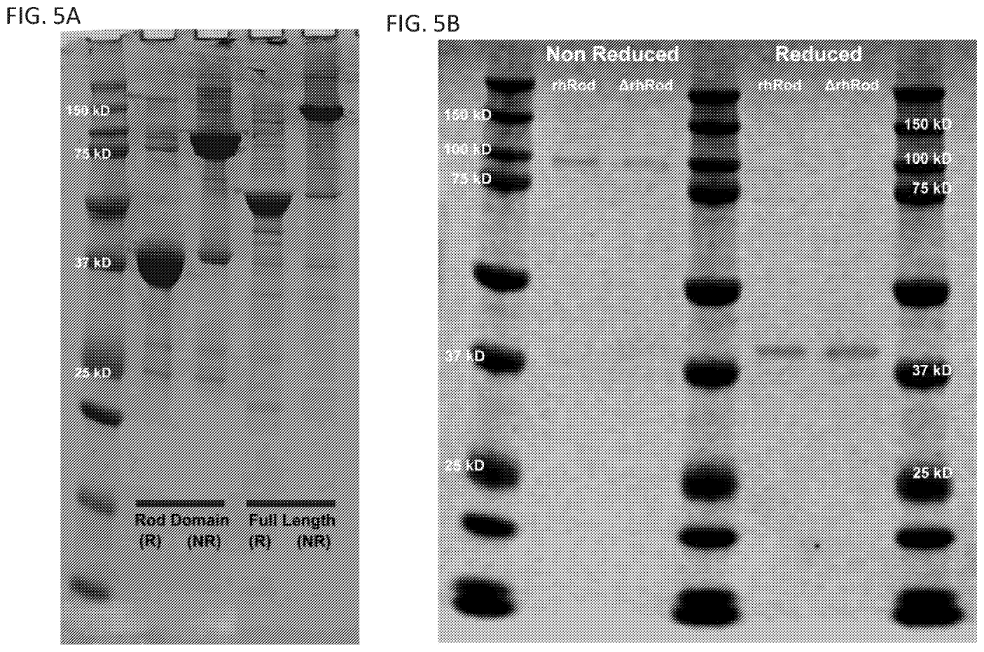

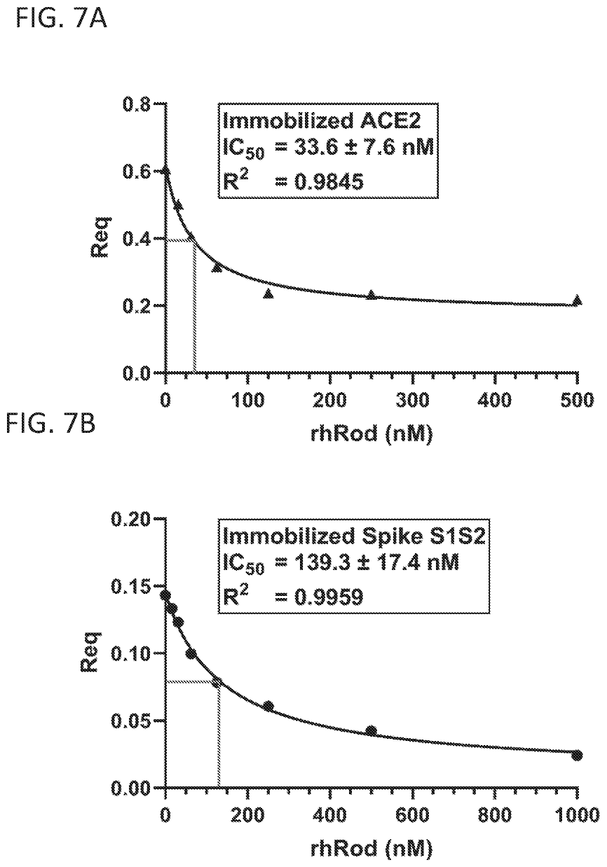

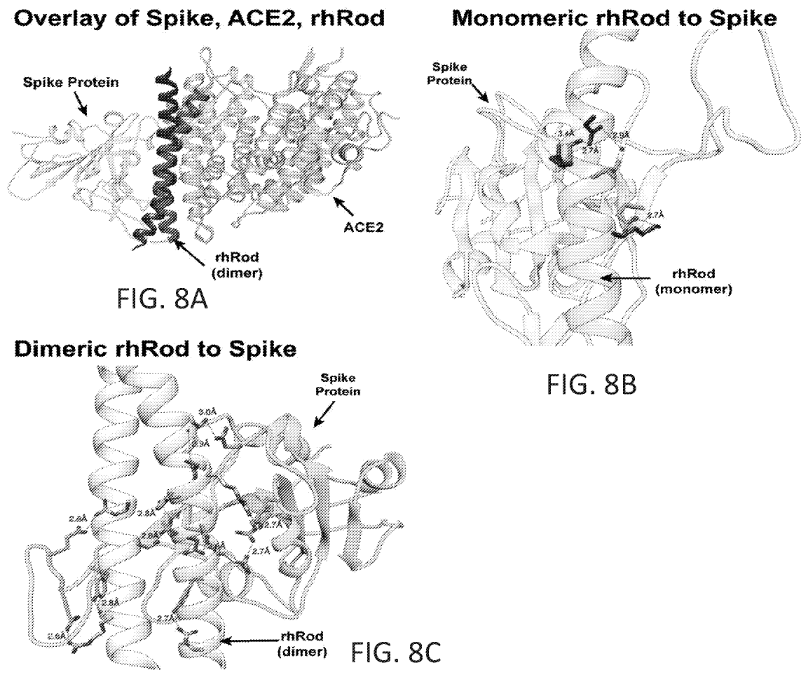

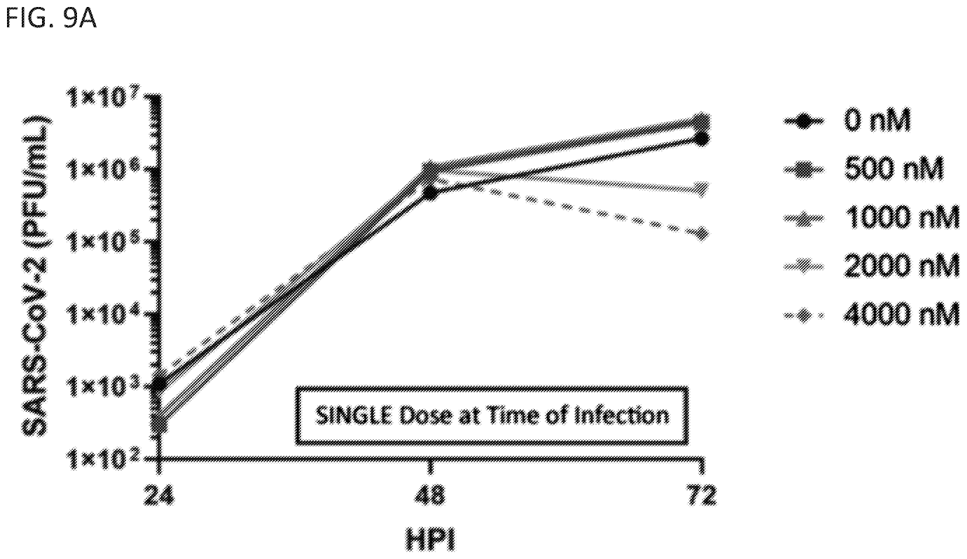

SARS-CoV-2 S shares ˜75% amino acid sequence identity as the SARS-CoV S, which binds to cell surface vimentin to enter host cells. This similarity makes it plausible that SARS-CoV-2 S would also bind to vimentin. The development of both human recombinant vimentin (rhVim) (Lam et al., J Immunol. 2018; 200(5):1718-26) and recombinant human rod domain of vimentin (rhRod) (Lam et al., PLoS ONE. 2020; 15(10)) is known in Escherichia coli to block leukocyte adhesion (FIG. 5). rhRod, but not recombinant full length vimentin, binds to spike protein (FIG. 6) to block spike-ACE2 interactions (FIG. 7). In silico modeling suggests potential epitopes within both rhRod and spike on where the interactions may occur (FIG. 8). One can identify the key amino acid residues within each epitope and generate positional scanning libraries to gain biochemical insight on the mechanism of rhRod-spike protein interactions and to generate novel peptide sequences of vimentin that have enhanced binding to spike protein.

Generation and testing of rhRod: It is known that both recombinant full length vimentin (rhVim) and the rod domain of vimentin (rhRod; aa 96-407 of full length vimentin) may be produced in Escherichia coli (Lam et al., 2020) and their ability to bind to P-selectin with high affinity to block P-selectin-PSGL-1 interactions. Both rhVim and rhRod blocked leukocyte adhesion to inflamed endothelium both in vitro and in vivo. FIG. 5 shows Coomassie blue staining of both reduced and non-reduced rhRod and rhVim (full length; FIG. 5A). The non-reduced form of rhRod is twice the size of the reduced form, suggesting that rhRod naturally forms dimers. In some studies, there was heat-inactivated rhRod (ΔrhRod; comparing to intact rhRod) to test whether the tertiary structure was necessary for its effect. There were no differences in migration size between rhRod and ΔrhRod in both non-reduced and reducing conditions (FIG. 5B) (Lam et al., 2020).

rhRod, but not recombinant full-length vimentin, binds to immobilized spike (S) protein: recombinant S protein (S1S2ECD-His; Sino Biological) was immobilized to AR2G biosensors (Forte Bio). Increasing concentrations of rhRod were added into different wells to create steady state binding curves using biolayer interferometry (BLI; Octet Red384) to determine the KD (FIGS. 6A & 6B). rhRod bound to immobilized S. Intact rhRod is necessary, as heat inactivated rhRod (ΔrhRod) did not bind to immobilized S protein (KD indeterminate; FIGS. 6C & 6D). This was replicated with multiple, separate lots of rhRod with similar results (FIG. 6E), demonstrating that the observation is not lot-dependent. rhRod was compared to recombinant full length vimentin (rhVim) in their ability to bind to immobilized S protein, and rhVim did not bind to spike protein (performed in duplicate; FIG. 6F). In specific embodiments, this may stem from the fact that the head domain of vimentin binds to the α-helical rod domain of vimentin (Traub et al., J Cell Sci. 1992; 101 (Pt 2):363-81). This indicates that the rod domain is the primary binding domain to S protein and that the head and/or tail domains may inhibit recombinant vimentin's binding to spike protein.

rhRod blocks spike protein binding to immobilized ACE2: Follow up experiments were performed to determine whether rhRod blocks spike protein binding to ACE2 protein. First was measured the K_{D} of spike protein (130±20 nM) and rhRod (780±210 nM) to AR2G-sensor immobilized human recombinant ACE2 protein (Sigma). Then, 125 nM of spike protein was mixed with increasing concentrations of rhRod prior to addition into wells for BLI analysis. By plotting the binding response against the rhRod concentration, it was found that increasing concentrations of rhRod block spike protein binding to immobilized ACE2 (IC50 33.6±7.6 nM; R2 0.9845; FIG. 7A). Because there may be differences in how proteins interact with the biosensors during immobilization, the experiment was repeated with immobilization of spike protein (S1S2). In this next set of studies, immobilized spike protein was first placed into wells with increasing concentrations of rhRod. After weakly/unbound rhRod was dissociated, sensors were placed into a second well with a constant concentration of ACE2 (200 nM). The ACE2 response was then measured and used to calculate the IC50 of rhRod to block spike-ACE2 interactions. Similar to the previous blocking experiment, rhRod blocked ACE2 binding to immobilized spike protein (IC₅₀ 139.3±17.4 nM; R² 0.9959; FIG. 7B).

In silico prediction model: SwarmDock was used to predict the interactions of the spike, ACE2, and rod domain of vimentin proteins (FIG. 8). rhRod may be blocking ACE2-spike protein interactions through mimicking the α-helix structure of ACE2 (FIG. 8A). Spike protein is predicted to form multiple hydrogen bonds and salt bridges with rhRod in both the monomeric (FIG. 8B) and dimeric (FIG. 8C) states, with more interactions between spike and dimeric rhRod (natural state)—suggesting a tighter bond with dimeric rhRod.

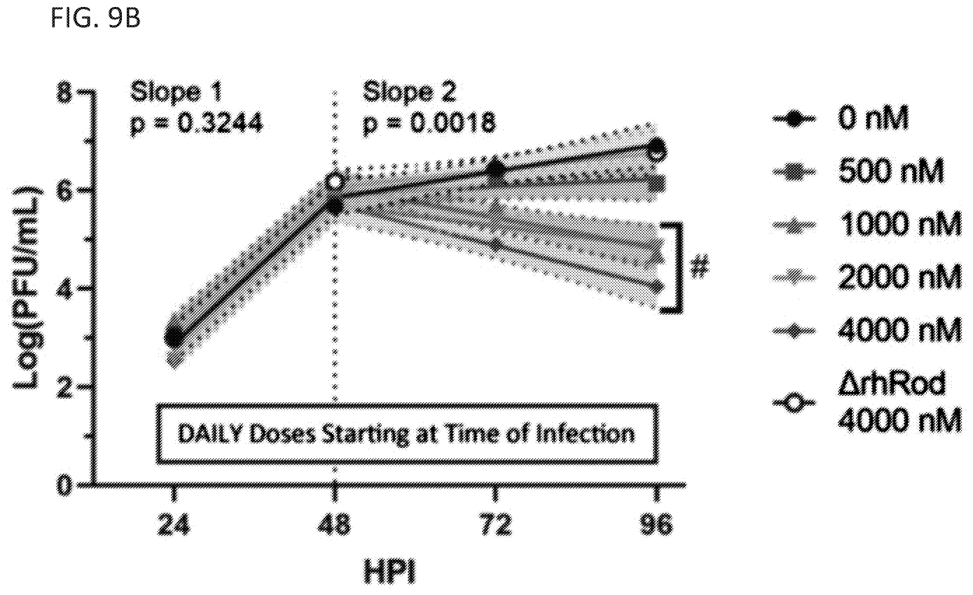

rhRod decreases SARS-CoV-2 replication and infectivity in Vero E6 cells: The efficacy was tested of rhRod to block infection of Vero E6 cells (Cercopithecus aethiops, Vero 76, clone E6, ATCC CRL-1586) by live, native SARS-CoV-2 (USA-WA1/2020). A single dose of rhRod (2,000-4,000 nM) or buffer control at time 0 into wells with Vero E6 cells and SARS-CoV-2 (MOI 0.1) reduced early viral replication (FIG. 9A). The experiments were repeated with doses (0-4,000 nM) given daily. As a secondary control, the highest dose of rhRod was heat inactivated (4,000 nM; BOILED) to determine whether the tertiary structure of rhRod was necessary to exert its effect on SARS-CoV-2 binding. When given daily, rhRod (1,000-4,000 nM) decreases viral replication by at least 100-fold (FIG. 9B). Heat inactivated rhRod (4,000 nM) had no effect, suggesting that the tertiary structure of rhRod was necessary to block SARS-CoV-2 viral replication.

### Examples of Specific Experimental Procedures

Identify the epitopes within rhRod and SARS-CoV-2 spike protein that form their interaction using reciprocal SPOT peptide arrays. One can generate SPOT peptide arrays of rhRod and spike protein, similar to our previous publication. Peptides of 20 amino acids (aa) in length with frame shifts of 2 aa may be placed per spot on a derivatized cellulose membrane using the MultiPep RS automated peptide synthesizer. Thus, each spot will have 18 aa of overlap between adjacent spots. Once the membranes are made, binding is detected using fluorescently labeled S protein (to bind to the rhRod SPOT array) or rhRod (to bind to the S protein SPOT array) with detection on a Li-Cor Odyssey Fc imaging system. These are performed in duplicate to ensure consistency.

Perform alanine scanning of rhRod epitopes to identify key amino acid residues. Once the epitopes on rhRod have been identified one can perform alanine scanning of candidate regions by substituting an alanine for each sequential non-alanine amino acid, one residue at a time. One can then compare binding of IR800 labeled spike protein of modified peptides to the original sequence. Decreases in fluorescence intensity after alanine-substitution will signify key residues within each spot.

Generate positional scanning libraries of key amino acid residues to assess the biochemical mechanism that supports rhRod-spike protein interactions. Once the key amino acid residues within each rhRod epitope are identified, one can perform positional scanning by substituting each key residue with all natural amino acids, one at a time. Changes in fluorescence intensity at each spot will signify an increased or decreased affinity of the modified rhRod sequence to IR800 labeled spike protein.

Compare the KD of candidate novel peptides (versus rhRod) and spike protein using biolayer interferometry. After candidate sequences are identified, one can create His-tagged peptides in E. coli and test their KD to spike protein such as in FIG. 6. Peptides whose affinity to S protein is equal to or higher than rhRod's affinity to S protein are used to measure their IC50 in blocking S protein-ACE2 interactions, similar to data in FIG. 7.

Measure the efficacy of rhRod and candidate novel peptides in reducing SARS-CoV-2 replication in Vero E6 and human pulmonary cell lines. Experiments are performed similar to those in FIG. 9 within a BSL-3 facility. One can culture Vero E6, human pulmonary airway epithelial cells, pulmonary microvascular endothelial cells, and pulmonary artery endothelial cells in multi-well plates. Cells are infected with SARS-CoV-2 (USA-WA1/2020 or a newly emerged variant strain; MOI 0.1) and then treated with rhRod or candidate peptide in a wide range of concentrations (0-16,000 nM) twice daily for up to 1 week. In addition to vehicle control, one can include heat inactived rhRod (ΔrhRod) as well as rhVim, since neither ΔrhRod nor rhVim bound to SARS-CoV-2 S protein (FIG. 3). One can measure cytopathogenicity and viral copy numbers using the xCELLigence Real-Time Cell Analysis system. Viral titers at designated time points are confirmed by plaque assay. One can use a super-resolution laser scanning confocal microscope (Olympus FV3000) to perform immunofluorescence microscopy on fixed samples to evaluate for binding and localization (cell surface versus intracellular) of rhRod/peptides (anti-His Ab) and virus (anti-spike Ab) on host cells relative to each other and cell surface vimentin (anti-vimentin Ab) and ACE2 (anti-ACE2 Ab).

Compare the efficacy of candidate novel peptides to rhRod (and control) to block SARS-CoV-2 infection of human airway organoids (HAO). One can confirm findings referred to above on human airway organoids (HAO). The organoid model allows evaluation of rhRod in a more complete system with cell differentiation.

Evaluate the efficacy of rhRod and candidate novel peptides in SARS-CoV-2-infected K18-hACE2 mice. The K18-hACE2 mouse (Jackson Laboratories; B6.Cg-Tg(K18-ACE2)2Prlmn/J; #034890) is susceptible to SARS-CoV-2 due to the presence of human ACE2 on epithelia. These mice develop signs and symptoms of COVID-19. One can intranasally inoculate male and female mice (12-16 weeks old) with 104 PFU of SARS-CoV-2 (USA-WA1/2020 versus variant strain) or saline control. Based on in vitro data (FIG. 9), mice will then be treated intranasally twice daily for up to 1 week with one of the following proteins/doses:
rhRod: 0 μM (vehicle control), 2, 4, 8, or 12 μM
ΔrhRod (negative control): 12 μM
rhVim (negative control since it did not bind to spike protein in our preliminary data [FIG. 6F]): 12 μM
Candidate Peptides (as they are identified and first tested in vitro): 0 μM, 2 μM, 4 μM, 8 μM, 12 μM

In some embodiments, multiple doses may be necessary to completely saturate SARS-CoV-2 S protein accounting for multiple S proteins on each virion and heterogeneity in drug delivery and viral replication phase. Mice in initial experiments will be treated with drug at the time of infection (t0). Once optimal dosing has been established, one can evaluate the efficacy of preventive and delayed drug administration relative to the onset of infection (t0) to better replicate clinical scenarios: t0-1 hour (preventive), t0+12 hours, t0+24 hours, t0+48 hours, and an untreated but infected group.

One can measure clinical activity using the mouse clinical assessment score for sepsis (which includes weight, activity, posture, behavior, appearance, respirations, and chest sounds) 23, 55. At the end of 1 week, mice are euthanized. Arterial blood gases, rodent blood panels and chemistries (to evaluate biochemical markers for organ injury), and plasma are collected during a terminal blood collection. After euthanasia, organs (lungs, kidney, liver, brain, and spleen) are collected and processed for histology (hematoxylin and eosin, immunohistochemistry, and immunofluorescence) and flash frozen samples are collected to evaluate for protein and RNA measurements of virus, inflammatory cytokines, as well as for use in determining viral titers by plaque assay.

Assess the immunogenicity of intranasal rhRod and candidate novel peptides in uninfected K18-hACE2 mice. One can evaluate whether mice receiving rhRod and candidate peptides develop immune responses to these proteins. One can treat male and female mice (12-16 weeks old) with rhRod/candidate novel peptides or vehicle control for 1 week based on the optimal dosing identified as described elsewhere herein. After 1 week of treatment, mice are observed for 28 days before terminal blood collection. At the conclusion of the study, plasma levels of total IgG, anti-rhRod, and anti-peptide antibodies are measured using ELISA and biolayer interferometry. After euthanasia, organs are collected to evaluate for potential organ injury or immune complex deposition related to drug therapy.

### Embodiments Wherein rhRod Improves COVID-19 by Blocking Inflammation Via P-Selectin

One can study the effect of rhRod on attenuating inflammation through blocking leukocyte infiltration into the lungs and other organs via blocking P-selectin-PSGL-1 interactions. In specific embodiments, rhRod has beneficial effects via two separate mechanisms; therefore, one can compare the effect of intraperitoneal (systemic) rhRod alone, intranasal (local) rhRod alone, and combined dosing. To assess P-selectin's role in the development of COVID-19 as well as rhRod's potential therapeutic effects, one can generate P-selectin−/−/K18-hACE2 mice (and their littermate controls). Finally, one can identify the key amino acid residues within rhRod that confer binding to P-selectin.

The role of P-selectin on leukocyte recruitment into the lungs and the development of acute respiratory distress syndrome is known. rhRod blocks leukocyte adhesion to inflamed endothelium and platelets similarly to rhVim. Furthermore, rhRod colocalizes with P-selectin and decreases neutrophil adhesion without changing blood flow velocity. Therefore, the ability of rhRod to block pathologic inflammation and reduce severity of COVID-19 via blocking P-selectin is characterized.

P-selectin deficient (P-sel−/−) mice have decreased neutrophil extravascular accumulation across inflamed endothelium: P-selectin has a role on neutrophil transmigration using a corneal abrasion model of sterile inflammation. This model produces an intense leukocytic infiltrate to the injured central cornea. As compared to wildtype mice, P-sel−/− mice had decreased neutrophil extravasation after corneal injury.

Both rhVim and rhRod bind to P-selectin to block P-selectin-PSGL-1 interactions and attenuate leukocyte recruitment during inflammation: rhVim binds to P-selectin and blocks leukocyte recruitment to inflamed endothelium and platelets in vitro and decreased leukocyte recruitment in lungs of endotoxemic mice. Using SPOT peptide arrays, the rod domain was identified as the region within vimentin which binds P-selectin (Lam et al., 2020). Based on those data, rhRod was generated and it was found that it binds specifically to P-selectin (Lam et al., 2020). Like rhVim, rhRod blocks leukocyte adhesion to platelets and inflamed endothelium in vitro (Lam et al., 2020). Finally, using intravital microscopy to assess leukocyte recruitment in a living animal, rhRod colocalized with P-selectin in the hepatic sinusoids and decreased neutrophil accumulation in endotoxemic mice (Lam et al., 2020). It was considered that rhRod may decrease leukocytic infiltration into the lungs and the severity of COVID-19 in K18/hACE2 mice.

Evaluate the efficacy of systemic (intraperitoneal) rhRod on COVID-19 severity in K18-hACE2 mice. K18-hACE2 male and female mice are inoculated with 104 PFU of SARS-CoV-2 (USA-WA1/2020 and a new variant) or saline. To determine the efficacy of systemic rhRod, one can inject one of the following intraperitoneally as a single dose at the time of infection (t0):
rhRod: 0 (vehicle control), 3, 6, and 10 mg/kg
ΔrhRod (negative control): 10 mg/kg
Candidate Peptides (as they are identified and tested first in vitro): 0, 3, 6, and 10 mg/kg

These doses were based on our published data in endotoxemic mice in which we gave 3 mg/kg rhVim (Lam et al., 2018) or rhRod (Lam et al., 2020) as a single dose to block leukocyte adhesion. A heat-inactivated dose was included of rhRod (ΔrhRod) at the highest dose, similar to in our preliminary in vitro studies (FIGS. 6 and 9), to serve as an additional control. Mice in initial experiments are treated with drug at the time of infection (t0). Once optimal dosing has been established, one can evaluate the efficacy of preventative and delayed drug administration relative to the onset of infection (t0) to better replicate clinical scenarios: t0-1 hour (preventive), t0+12 hours, t0+24 hours, t0+48 hours, and an untreated infected control group.

One can measure clinical activity using a mouse clinical assessment score for sepsis (which includes weight, activity, posture, behavior, appearance, respirations, and chest sounds). At the end of 1 week, mice are anesthetized and arterial blood gases, rodent blood panels and chemistries (to evaluate biochemical markers for organ injury) and plasma are collected during a terminal blood collection. After euthanasia, organs (lungs, kidney, liver, brain, and spleen) are collected and processed for histology (H&E, immunohistochemistry, and immunofluorescence) and flash frozen samples are collected to evaluate for protein and RNA measurements of virus and inflammatory cytokines.

Compare the efficacy of combined (intranasal+intraperitoneal) to either intranasal or intraperitoneal rhRod alone on COVID-19 severity in K18-hACE2 mice. Although rhRod binds to the spike protein and decreases viral replication (FIG. 9), the doses given may not fully block all spike protein sites of all viruses in the body. Furthermore, patients with COVID-19 may also develop systemic effects and pathologic inflammation. Therefore, there may be a need for multiple routes of administration based on rhRod's ability to block both viral adhesion and leukocyte recruitment. Systemic (intraperitoneal) rhRod may be beneficial to attenuate pathologic inflammation while additional intranasal rhRod is used to completely block spike protein within the airways. One can determine if combined administration of rhRod is synergistic as compared to either route alone, using the dosing regimen as described above (intranasal or intraperitoneal) and one can compare combination therapy (intranasal+intraperitoneal) to each route alone after inoculation with 104 PFU of SARS-CoV-2:
Intranasal (rhRod)+Intraperitoneal (control)
Intranasal (control)+Intraperitoneal (rhRod)
Intranasal (rhRod)+Intraperitoneal (rhRod)
Intranasal (control)+Intraperitoneal (control)

Analysis of the efficacy of each route may be similar to procedures described above. As candidate novel peptides are discovered and systematically tested in vitro, they are included in these studies.

Test the efficacy of systemic rhRod on COVID-19 in P-selectin−/−/K18-hACE2 mice. Since P-selectin may play a role in the development of COVID-19, it is considered that P-selectin−/−/K18-hACE2 mice have decreased COVID-19 severity. Since rhRod binds to P-selectin to block P-selectin-PSGL-1 interactions, it is considered that systemic rhRod will not further decrease COVID-19 severity in P-selectin−/−/K18-hACE2 mice, but rhRod will benefit P-selectin+/+/K18-hACE2 mice. One can cross P-selectin−/− and K18-hACE2 mice to create P-selectin−/−/K18-hACE2 (Psel−/K18) and P-selectin+/+/K18-hACE2 (Psel+/K18) mice. One can inoculate mice with 104 PFU of SARS-CoV-2 (USA-WA1/2020). Based on the optimal dose determined as described above, one can compare the effect of intraperitoneal rhRod versus control on the severity of COVID-19 in Psel-/K18 mice as compared to Psel+/K18 mice:
Psel⁻/K18: Control or rhRod
Psel⁺/K18: Control or rhRod

Analysis of the efficacy of rhRod in the absence of P-selectin (Psel−/K18) is similar to studies described above.

Perform alanine scanning of rhRod epitopes to identify key amino acid residues which bind to P-selectin. One can perform alanine scanning on the epitopes identified on rhRod that correlated with its ability to bind to P-selectin. One can then compare binding of IR800 labeled P-selectin of modified peptides to the original sequence. Decreases in fluorescence intensity after alanine-substitution signifies key residues within each spot.

Create positional scanning libraries of key amino acid residues to assess the biochemical mechanism that supports rhRod-spike protein interactions. Once the key amino acid residues within each rhRod epitope have been identified, one can perform positional scanning by substituting each key residue with all natural amino acids, one at a time. Changes in fluorescence intensity at each spot will signify an increased or decreased affinity of the modified rhRod sequence to IR800 labeled spike protein. Candidate novel peptides are generated.

Measure the KD of candidate novel peptides binding to P-selectin and their IC50 to block P-selectin-PSGL-1 interactions using biolayer interferometry. Peptides identified as described elsewhere herein are generated and tested in an OctetRed384 biolayer interferometry system to measure the KD to P-selectin and the IC50 to PSGL-1, similar to FIGS. 6 & 7.

Compare candidate novel peptides on blocking leukocyte adhesion to P-selectin, platelet monolayers, and inflamed endothelial monolayers in vitro using the Bioflux microfluidic parallel plate flow system. After identifying candidate peptides, one can compare the peptides to rhRod (at increasing concentrations) on blocking human whole blood leukocyte and isolated neutrophil adhesion over P-selectin-coated channels, platelet monolayers, and endothelial monolayers under physiologic shear (2 dyn/cm²).

rhRod and Peptide Production: rhRod may be produced in E. coli (strain M15) transformed with a pQE-30 vector containing the sequence encoding the rod domain of vimentin (NM_00380.3 [NCBI]; bp 717-1,652; aa residues 96-407). After protein expression is induced, the bacteria are pelleted and lysed. Bacterial lysates are centrifuged and the supernatants collected and filtered prior to loading onto nickel affinity columns to capture the His-tagged proteins. Bound protein is then eluted with imidazole and the purified samples serially dialyzed to reduce the urea concentration. rhRod is ultimately dialyzed against 20 mM sodium phosphate buffer, pH 8, prior to sterile filtration using a 0.22 μm filter under sterile conditions. Absorbance is measured at 280 nm with protein concentrations determined using a molecular weight 38.2 kD and extinction coefficient of 15,350 L/mol/cm. Purity is confirmed using SDS-PAGE and Coomassie blue stain and western blot with anti-vimentin antibodies. Multiple lots are created with retransforming E. coli to ensure that the effects seen are not lot dependent. Candidate peptides are produced in a similar manner as above.

SARS-CoV-2 Production: SARS-CoV-2 (USA-W A1/2020) was obtained from the University of Texas Medical Branch World Reference Center for Emerging Viruses and Arboviruses, and SARS-CoV-2 (B.1.1.7) was obtained from BEI Resources. The virus was grown on Vero CCL81 (ATCC® CCL-81™) and titers were determined by standard plaque assay on Vero E6 (ATCC CRL-1586). After each passage, virus is sequenced and compared to the parent strain to ensure no changes have occurred that may alter the outcomes of the experiment. As new variant strains emerge, they can be obtained when they are available.

Colony Planning of P-selectin−/−/K18-hACE2 mice: A breeding colony of P-selectin−/− and K18-hACE2 mice are maintained. To create the P-selectin−/−/K18-hACE2 mice, one can generate mating pairs of homozygous mutants with homozygous mutants, homozygous mutants with heterozygous mutants, and heterozygous mutants with heterozygous mutants until a colony is established. Offspring from the second two mating schemes are genotyped to ensure the desired outcomes are present in offspring. Mating pairs may begin with mice that are between 6-12 weeks of age. Nonproductive breeders are replaced, and breeders are retired around 8 months of age.

Sample Size Calculation and Data Analysis: For in vitro studies, studies are performed in duplicate with at least 3 lots of rhRod to ensure that the results seen are not lot-specific. Based on data FIG. 6, an n=3 with an α probability of 0.05 and the same effect size would produce a power (1−β) of >0.99 when comparing the highest concentration to control. For the in vivo studies, a sample size of 6 per group (3 females and 3 males per group) may be used to observe a 30% decrease in the acute lung injury score with a standard deviation of 10% per group, an α=0.01, and β=0.1 between the highest rhRod dose and control. One can use conservative α- and β-values because of the nature of the sample size estimation using t-tests whereas the study can measure different doses, routes, and time points. Being more conservative with estimates can give more confidence to the post hoc analyses when comparing different doses and routes (using 2-way analysis of variance). Data analysis may be performed using GraphPad Prism software.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods, systems, and steps described in the specification. As one of ordinary skill in the art will readily appreciate from the present disclosure, processes, machines, manufacture, compositions of matter, means, methods, systems, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, systems, or steps.

### REFERENCES

1. Wang Q, Zhang Y, Wu L, Niu S, Song C, Zhang Z, Lu G, Qiao C, Hu Y, Yuen K Y, Wang Q, Zhou H, Yan J, Qi J. Structural and Functional Basis of SARS-CoV-2 Entry by Using Human ACE2. Cell. 2020. doi: 10.1016/j.cell.2020.03.045. PubMed PMID: 32275855; PMCID: PMC7144619.
2. Yan R, Zhang Y, Li Y, Xia L, Guo Y, Zhou Q. Structural basis for the recognition of SARS-CoV-2 by full-length human ACE2. Science. 2020; 367(6485):1444-8. doi: 10.1126/science.abb2762. PubMed PMID: 32132184.
3. Ou X, Liu Y, Lei X, Li P, Mi D, Ren L, Guo L, Guo R, Chen T, Hu J, Xiang Z, Mu Z, Chen X, Chen J, Hu K, Jin Q, Wang J, Qian Z. Characterization of spike glycoprotein of SARS-CoV-2 on virus entry and its immune cross-reactivity with SARS-CoV. Nat Commun. 2020; 11(1):1620. doi: 10.1038/s41467-020-15562-9. PubMed PMID: 32221306; PMCID: PMC7100515.
4. Yu Y T, Chien S C, Chen I Y, Lai C T, Tsay Y G, Chang S C, Chang M F. Surface vimentin is critical for the cell entry of SARS-CoV. J Biomed Sci. 2016; 23:14. doi: 10.1186/s12929-016-0234-7. PubMed PMID: 26801988; PMCID: PMC4724099.
5. Lam F W, Da Q, Guillory B, Cruz M A. Recombinant Human Vimentin Binds to P-Selectin and Blocks Neutrophil Capture and Rolling on Platelets and Endothelium. J Immunol. 2018; 200(5):1718-26. doi: 10.4049/jimmunol.1700784. PubMed PMID: 29335256; PMCID: PMC5821592.

What is claimed is:

1. A method of treating or preventing coronavirus infection in an individual, comprising the step of delivering to the individual a therapeutically effective amount of a composition comprising vimentin or a functionally active fragment or variant thereof.

2. The method of claim 1, wherein the coronavirus infection comprises SARS-CoV-2 infection.

3. The method of claim 1 or 2, wherein the individual has severe acute respiratory syndrome (SARS), COVID-19, MERS, or a respiratory infection.

4. The method of any one of the preceding claims, wherein the individual has fever, cough, shortness of breath or difficulty breathing, tiredness, aches, chills, sore throat, loss of smell, loss of taste headache, diarrhea, vomiting, pneumonia, acute respiratory distress syndrome, organ failure, respiratory failure, heart conditions such as cardiomyopathies, acute kidney injury, cognitive conditions, and/or further viral and bacterial infections, or a combination thereof.

5. The method of any one of the preceding claims, wherein the fragment of vimentin comprises N-terminal head domain, C-terminal tail domain, α-helical coiled-coil rod domain, or a combination thereof.

6. The method of any one of the preceding claims, wherein the fragment comprises the N-terminus, the C-terminus, both the N-terminus and C-terminus, or neither of the N-terminus or C-terminus.

7. The method of any one of the preceding claims, wherein the fragment or derivative is at least 70, 75, 80, 85, 90, 91, 92, 93, 94, 95, 96, 97, 98, or 99% identical to SEQ ID NO:1.

8. The method of any one of the preceding claims, wherein the variant comprises 1, 2, 3, 4, 5, or more variations compared to SEQ ID NO:1, SEQ ID NO:2, SEQ ID NO:4, or SEQ ID NO:6.

9. The method of any one of the preceding claims, wherein the fragment comprises SEQ ID NO:2.

10. The method of any one of the preceding claims, wherein the fragment or derivative is at least 70, 75, 80, 85, 90, 91, 92, 93, 94, 95, 96, 97, 98, or 99% identical to SEQ ID NO:2.

11. The method of any one of the preceding claims, wherein the variant comprises 1, 2, 3, 4, 5, or more variations compared to SEQ ID NO:2.

12. The method of any one of the preceding claims, wherein the fragment comprises a peptide sequence from SEQ ID NO:2.

13. The method of claim 12, wherein the peptide is at least or no more than 8, 9, 10, 11, 12, 13, 14, 15, 16, 17, 18, 19, 20, 21, 22, 23, 24, 25, 26, 27, 28, 29, 30, 31, 32, 33, 34,3 5, 36, 37, 38, 39, 40, 41, 42, 43, 44, 45, 46, 47, 48, 49, or 50 contiguous amino acids of SEQ ID NO:2.

14. The method of any one of the preceding claims, wherein the vimentin or a functionally active fragment or variant thereof binds a coronavirus spike protein.

15. The method of any one of the preceding claims, wherein the individual is provided an effective amount of a second therapy for the coronavirus infection.

16. The method of claim 15, wherein the second therapy comprises antibiotics, antivirals, convalescent serum, immune modulators, anticoagulants, fluids, oxygen, a corticosteroid, antibodies, GSnP-6, sialyl Lewis X analog, anti-proliferatives, calcineurin inhibitors, anti-signaling compounds, or a combination thereof.

17. The method of claim 15, wherein the second therapy comprises an anti-SARS-CoV-2 drug.

18. The method of claim 17, wherein the anti-SARS-CoV-2 drug is selected from the group consisting of Azithromycin, AC-55541, Apicidin, AZ3451, AZ8838, Bafilomycin A1, CCT 365623, Daunorubicin, E-52862, Entacapone, GB110, H-89, Haloperidol, Indomethacin, JQ1, Loratadine, Merimepodib, Metformin, Midostaurin, Migalastat, Mycophenolic acid, PB28, PD-144418, Ponatinib, Ribavirin, RS-PPCC, Ruxolitinib, RVX-208, S-verapamil, Silmitasertib, TMCB, UCPH-101, Valproic Acid, XL413, ZINC1775962367, ZINC4326719, ZINC4511851, ZINC95559591, 4E2RCat, ABBV-744, Camostat, Captopril, CB5083, Chloramphenicol, Chloroquine (and/or Hydroxychloroquine), CPI-0610, Dabrafenib, DBeQ, dBET6, IHVR-19029, Linezolid, Lisinopril, Minoxidil, ML240, MZ1, Nafamostat, Pevonedistat, PS3061, Rapamycin (Sirolimus), Sanglifehrin A, Sapanisertib (INK128/M1N128), FK-506 (Tacrolimus), Ternatin 4(DA3), Tigecycline, Tomivosertib (eFT-508), Verdinexor, WDB002, Zotatifin (eFT226), and a combination thereof.

19. The method of any one of the preceding claims, wherein the method further comprises testing for the coronavirus infection.

20. The method of any one of the preceding claims, wherein the coronavirus infection is prevented, reduced in severity, and/or delayed in onset.

21. The method of any one of the preceding claims, wherein the composition is delivered to the individual intravenously, intradermally, transdermally, intrathecally, intraarterially, intraperitoneally, intranasally, intravaginally, intrarectally, topically, intramuscularly, subcutaneously, mucosally, orally, topically, locally, by inhalation, by injection, by infusion, via catheter, and/or via lavage.

22. The method of any one of the preceding claims, wherein the composition is delivered to the individual multiple times.

23. The method of claim 22, wherein the composition is delivered to the individual once a day, more than once a day, more than once a week, more than once a month, or more than once a year.

24. The method of any one of the preceding claims, wherein the composition is provided to the individual by constant infusion.

25. A system for treating or preventing coronavirus infection in an individual, comprising a surface external to the individual that is coated with a composition comprising vimentin or a functionally active fragment or variant thereof.

26. The system of claim 25, wherein the fragment of vimentin comprises N-terminal head domain, C-terminal tail domain, α-helical coiled-coil rod domain, or a combination thereof.

27. The system of claim 25 or 26, wherein the fragment comprises the N-terminus, the C-terminus, both the N-terminus and C-terminus, or neither of the N-terminus or C-terminus.

28. The system of any one of claims 25-27, wherein the fragment or derivative is at least 70, 75, 80, 85, 90, 91, 92, 93, 94, 95, 96, 97, 98, or 99% identical to SEQ ID NO:1.

29. The system of any one of claims 25-28, wherein the variant comprises 1, 2, 3, 4, 5, or more variations compared to SEQ ID NO:1, SEQ ID NO:2, SEQ ID NO:4, or SEQ ID NO:6.

30. The system of any one of claims 25-28, wherein the fragment comprises SEQ ID NO:2.

31. The system of any one of claims 25-30, wherein the fragment or derivative is at least 70, 75, 80, 85, 90, 91, 92, 93, 94, 95, 96, 97, 98, or 99% identical to SEQ ID NO:2.

32. The system of any one of claims 25-31, wherein the variant comprises 1, 2, 3, 4, 5, or more variations compared to SEQ ID NO:2.

33. The system of any one of claims 25-32, wherein the composition binds a coronavirus spike protein.

34. The system of any one of claims 25-33, wherein the surface is in or on a membrane, tube, filter, or container.

35. The system of claims 25-34, wherein the system further comprises a test for the coronavirus infection.

36. The system of claims 25-35, wherein the system further comprises an effective amount of a second therapy for a coronavirus infection.

37. The system of claim 36, wherein the second therapy comprises antibiotics, antivirals, convalescent serum, immune modulators, anticoagulants, fluids, oxygen, a corticosteroid, antibodies, GSnP-6, sialyl Lewis X analog, anti-proliferatives, calcineurin inhibitors, anti-signaling compounds, or a combination thereof.

38. The system of claim 36 or 37, wherein the second therapy comprises an anti-SARS-CoV-2 drug.

39. The system of claim 38, wherein the anti-SARS-CoV-2 drug is selected from the group consisting of Azithromycin, AC-55541, Apicidin, AZ3451, AZ8838, Bafilomycin A1, CCT 365623, Daunorubicin, E-52862, Entacapone, GB110, H-89, Haloperidol, Indomethacin, JQ1, Loratadine, Merimepodib, Metformin, Midostaurin, Migalastat, Mycophenolic acid, PB28, PD-144418, Ponatinib, Ribavirin, RS-PPCC, Ruxolitinib, RVX-208, S-verapamil, Silmitasertib, TMCB, UCPH-101, Valproic Acid, XL413, ZINC1775962367, ZINC4326719, ZINC4511851, ZINC95559591, 4E2RCat, ABBV-744, Camostat, Captopril, CB5083, Chloramphenicol, Chloroquine (and/or Hydroxychloroquine), CPI-0610, Dabrafenib, DBeQ, dBET6, IHVR-19029, Linezolid, Lisinopril, Minoxidil, ML240, MZ1, Nafamostat, Pevonedistat, PS3061, Rapamycin (Sirolimus), Sanglifehrin A, Sapanisertib (INK128/M1N128), FK-506 (Tacrolimus), Ternatin 4 (DA3), Tigecycline, Tomivosertib (eFT-508), Verdinexor, WDB002, Zotatifin (eFT226), and a combination thereof.

40. A method for treating or preventing coronavirus infection in an individual, comprising the step of subjecting the blood or plasma of an individual to the system of any one of claims 25-39.

41. The method of claim 40, wherein the coronavirus infection is prevented, reduced in severity, and/or delayed in onset in the individual.

42. The method of claim 40 or 41, wherein the individual is administered an effective amount of a second therapy.

43. A kit for treating or preventing coronavirus infection in an individual, comprising a composition comprising vimentin or a functionally active fragment or variant thereof and a second therapy for coronavirus infection or prevention, said composition and second therapy housed in one or more suitable containers.

44. The kit of claim 43, wherein the fragment of vimentin comprises N-terminal head domain, C-terminal tail domain, α-helical coiled-coil rod domain, or a combination thereof.

45. The kit of claim 43 or 44, wherein the fragment comprises the N-terminus, the C-terminus, both the N-terminus and C-terminus, or neither of the N-terminus or C-terminus.

46. The kit of any one of claims 43-45, wherein the fragment or derivative is at least 70, 75, 80, 85, 90, 91, 92, 93, 94, 95, 96, 97, 98, or 99% identical to SEQ ID NO:1.

47. The kit of any one of claims 43-46, wherein the variant comprises 1, 2, 3, 4, 5, or more variations compared to SEQ ID NO:1, SEQ ID NO:2, SEQ ID NO:4, or SEQ ID NO:6.

48. The kit of any one of claims 43-47, wherein the fragment comprises amino SEQ ID NO:2.

49. The kit of any one of claims 43-48, wherein the fragment or derivative is at least 70, 75, 80, 85, 90, 91, 92, 93, 94, 95, 96, 97, 98, or 99% identical to SEQ ID NO:2.

50. The kit of any one of claims 43-49, wherein the variant comprises 1, 2, 3, 4, 5, or more variations compared to SEQ ID NO:2.

51. The kit of any one of claims 43-50, wherein the second therapy comprises antibiotics, antivirals, convalescent serum, immune modulators, anticoagulants, fluids, oxygen, a corticosteroid, antibodies, GSnP-6, sialyl Lewis X analog, anti-proliferatives, calcineurin inhibitors, anti-signaling compounds, or a combination thereof.

52. The kit of any one of claims 43-51, wherein the second therapy comprises an anti-SARS-CoV-2 drug.

53. The kit of claim 52, wherein the anti-SARS-CoV-2 drug is selected from the group consisting of Azithromycin, AC-55541, Apicidin, AZ3451, AZ8838, Bafilomycin A1, CCT 365623, Daunorubicin, E-52862, Entacapone, GB110, H-89, Haloperidol, Indomethacin, JQ1, Loratadine, Merimepodib, Metformin, Midostaurin, Migalastat, Mycophenolic acid, PB28, PD-144418, Ponatinib, Ribavirin, RS-PPCC, Ruxolitinib, RVX-208, S-verapamil, Silmitasertib, TMCB, UCPH-101, Valproic Acid, XL413, ZINC1775962367, ZINC4326719, ZINC4511851, ZINC95559591, 4E2RCat, ABBV-744, Camostat, Captopril, CB5083, Chloramphenicol, Chloroquine (and/or Hydroxychloroquine), CPI-0610, Dabrafenib, DBeQ, dBET6, IHVR-19029, Linezolid, Lisinopril, Minoxidil, ML240, MZ1, Nafamostat, Pevonedistat, PS3061, Rapamycin (Sirolimus), Sanglifehrin A, Sapanisertib (INK128/M1N128), FK-506 (Tacrolimus), Ternatin 4 (DA3), Tigecycline, Tomivosertib (eFT-508), Verdinexor, WDB002, Zotatifin (eFT226), and a combination thereof.

54. The kit of any one of claims 43-53, wherein the composition further comprises a pharmaceutically acceptable carrier.


SEQUENCE LISTING (7 sequences)

SEQ ID NO: 1 - protein, 466 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 2 - protein, 312 aa, Homo sapiens
FKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGE

SEQ ID NO: 3 - DNA, 936 bp, Homo sapiens
ttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctgggggacctctacgaggaggagatgcgggagctgcgccggcaggtggaccagctaaccaacgacaaagcccgcgtcgaggtggagcgcgacaacctggccgaggacatcatgcgcctccgggagaaattgcaggaggagatgcttcagagagaggaagccgaaaacaccctgcaatctttcagacaggatgttgacaatgcgtctctggcacgtcttgaccttgaacgcaaagtggaatctttgcaagaagagattgcctttttgaagaaactccacgaagaggaaatccaggagctgcaggctcagattcaggaacagcatgtccaaatcgatgtggatgtttccaagcctgacctcacggctgccctgcgtgacgtacgtcagcaatatgaaagtgtggctgccaagaacctgcaggaggcagaagaatggtacaaatccaagtttgctgacctctctgaggctgccaaccggaacaatgacgccctgcgccaggcaaagcaggagtccactgagtaccggagacaggtgcagtccctcacctgtgaagtggatgcccttaaaggaaccaatgagtccctggaacgccagatgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacctacaggaagctgctggaaggcgag

SEQ ID NO: 4 - protein, 82 aa, Homo sapiens
MFGGPGTASRPSSSRSYVTTSTRTYSLGSALRPSTSRSLYASSPGGVYATRSSAVRLRSSVPGVRLLQDSVDFSLADAINTE

SEQ ID NO: 5 - DNA, 282 bp, Homo sapiens
tccaccaggtccgtgtcctcgtcctcctaccgcaggatgttcggcggcccgggcaccgcgagccggccgagctccagccggagctacgtgactacgtccacccgcacctacagcctgggcagcgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgag

SEQ ID NO: 6 - protein, 59 aa, Homo sapiens
ESRISLPLPNFSSLNLRETNLDSLPLVDTHSKRTLLIKTVETRDGQVINETSQHHDDLE

SEQ ID NO: 7 - DNA, 180 bp, Homo sapiens
gagagcaggatttctctgcctcttccaaacttttcctccctgaacctgagggaaactaatctggattcactccctctggttgatacccactcaaaaaggacacttctgattaagacggttgaaactagagatggacaggttatcaacgaaacttctcagcatcacgatgaccttgaataa